(19) United States
(12) Patent Application Publication
(10) Pub. No.: US 2019/0275027 A1
(43) Pub. Date: Sep. 12, 2019

(54) Bis-Benzyl-Tetrahydroisoquinoline Derivatives As Therapeutics For Filovirus

(71) Applicant: Southwest Research Institute, San Antonio, TX (US)

(72) Inventors: William E. BAUTA, San Antonio, TX (US); Jonathan A. BOHMANN, San Antonio, TX (US); Michael W. TIDWELL, Lakehills, TX (US); Andrey D. MALAKHOV, Helotes, TX (US); Alejandro SANTILLAN, JR., San Antonio, TX (US)

(21) Appl. No.: 15/916,596

(22) Filed: Mar. 9, 2018

(51) Int. Cl. A61K 31/4725; A61P 31/14

(52) U.S. Cl. CPC A61K 31/4725; A61P 31/14

(57) ABSTRACT

Bis-benzyl-tetrahydroisoquinoline analogs that are derivatives of the cyclic products tetrandrine (TETN) and cepharanthine (CEPH). The analogs indicate activity against filovirus infections, including the type species Marburg virus (MARV) and Ebola virus (EBOV).

### GOVERNMENT SUPPORT CLAUSE

This invention was made with government support under Contract No HHSN272201500015C awarded by the National Institutes of Health. The government has certain rights in the invention.

### FIELD OF THE INVENTION

The present invention is directed at a series of acyclic bis-benzyl-tetrahydroisoquinoline analogs that are derivatives of the cyclic products tetrandrine (TETN) and cepharanthine (CEPH). The analogs indicate activity against filovirus infections.

### BACKGROUND

Recent insights gained into the potential mode of action of the filovirus and its interaction with its host cells has identified several natural products that target the two pore channel (TPC2) that permits the release of v-RNA into the cell for further viral proliferation. The natural products tetrandrine (TETN) and cepharanthine (CEPH) have been shown to inhibit viral infections both in vitro and in vivo. Additionally, the antihypertensive properties of TETN and CEPH pose a challenge for drug candidates related to the natural products which dictates that they be evaluated for their selective inhibition of TPC2 versus other calcium channels. These finding led herein to the identification of structurally related analogs of TETN and CEPH with improved infectivity properties and diminished ion channel inhibition. It is contemplated that the drug candidates targeted will also possess improved aqueous solubility versus the natural products.

### SUMMARY

The present invention is directed at bis-benzyl-tetrahydroisoquinoline or a pharmaceutical salt thereof having the following Formula 1:

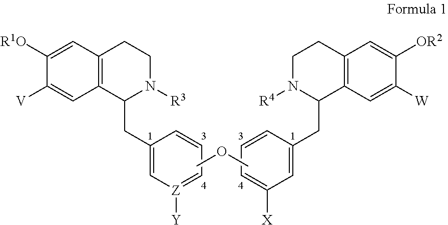

wherein R¹, R²=Me;

R¹ and V can combine to form a dioxolane ring,

R² and W can combine to form a dioxolane ring;

V, W═H, OMe;

R³, R⁴═H, Me, isopropyl, cyclopropyl, benzyl and C(O)—(CH₂)n-N-Me₂, n=1-3;

OR¹ can be replaced by a H;

X═H, OMe;

Z—Y═C—H, C—OMe, N; and

bi-aryl substitution: 1,4-1,4-; 1,4-1,3-; 1,3-1,3 isomers.

The present invention is also directed at a bis-benzyl-tetrahydroisoquinoline or a pharmaceutical salt thereof having the following Formula 2:

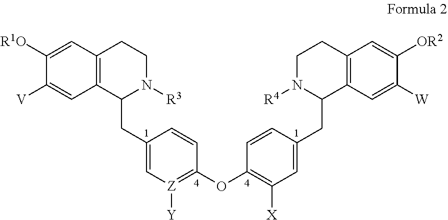

wherein R¹, R²=Me;
R¹ and V can combine to form a dioxolane ring;
R² and W can combine to form a dioxolane ring;
V, W═H, OMe;
OR¹ can be replaced by a H;
R³, R⁴═H, Me, isopropyl, cyclopropyl, benzyl and —C(O)—(CH₂)n-NMe₂, n=1-3;
X═H, OMe; and
Z—Y═C—H, C—OMe, N.

In yet still further embodiment, the present invention is directed at the following bis-benzyl-tetrahydroisoquinoline or a pharmaceutical salt thereof having the following Formula 3:

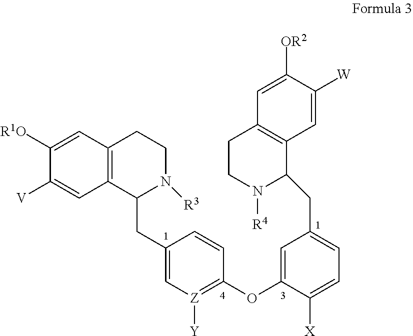

wherein R¹, R²=Me;
R¹ and V combine to form a dioxolane ring;
R² and W combine to form a dioxolane ring;
V, W═H, OMe;
R³, R⁴═H, Me, isopropyl, cyclopropyl, benzyl and —C(O)—(CH₂)n-NMe₂, n=1-3;
X═H, OMe;
Z—Y—C—H, C—OMe, N.

In a still further embodiment, the present invention is directed at the following bis-benzyl-tetrahydroisoquinoline or a pharmaceutical salt thereof having the following Formula 4:

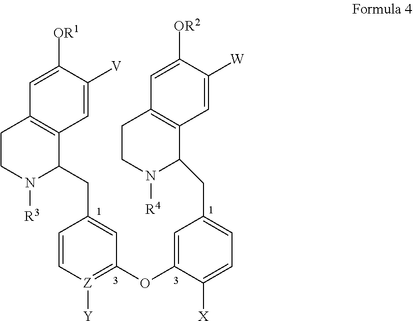

wherein R¹, R²=Me;
R¹ and V combine to form a dioxolane ring;
R² and W combine to form a dioxolane ring;
V, W═H, OMe;
R³, R⁴═H, Me, isopropyl, benzyl and C(O)—(CH₂)n-NMe₂, n=1-3;
X═H, OMe;
Z—Y═C—H, C—OMe, N.

In the above, reference to the dioxolane analog is reference to the placement of a dioxolane ring at the indicated location. For example, with reference to the first compound presented above, a dioxolane analog would have the following structure:

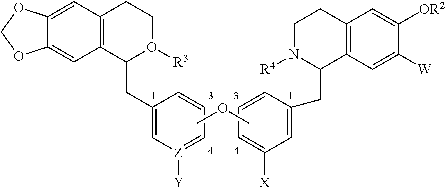

Furthermore, the present invention relates to a method of treating an individual infected with or exposed to a filovirus comprising administering to said individual as an active ingredient a compound of Formulas I, II, III or IV or a pharmaceutical salt thereof. The filovirus may include Ebola virus (EBOV) or Marburg virus (MARV).

### BRIEF DESCRIPTION OF THE DRAWINGS

FIG. 1 identifies the bis-N-methyl-tetrahydroisoquinoline analogs identified as 1, 2, 3 and the pyridiyl analogs identified as 4 and 5.

### DETAILED DESCRIPTION

As noted above, the present invention is directed to the above summarized bis-benzyl-tetrahydroisoquinoline analogs (Formulas 1, 2, 3 and 4) or a pharmaceutical salt thereof. Reference to a pharmaceutical salt is understood herein to be any one of Formulas 1, 2, 3 or 4 that has been combined with a counter-ion to form a neutral complex. Pharmaceutical salts herein therefore refers to salts of the Formulas 1, 2, 3 or 4 that are acceptable for clinical use. Methods for preparation of pharmaceutical salts are known in the art. For example, reference is made to the Handbook of Pharmaceutical Salts, Properties, Selection and Use, 2^{nd} Revised Edition, Wiley-VCG, P. H. Stahl and C. G. Wermuth (Editors), April 2011.

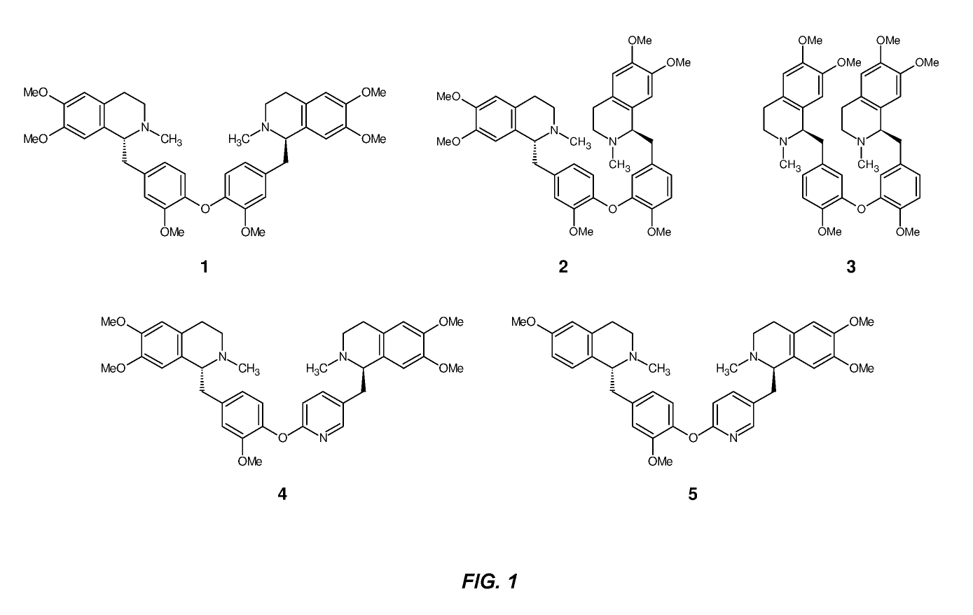

In order to demonstrate the synthesis of the bis-N-methyl-THIQ analogs, five targets (FIG. 1) are selected to describe their preparation. The analogs in FIG. 1 represent the parent compounds with the three substitution patterns on the linking biaryl ether unit: 1,4-1,4-, 1,4-1,3- and 1,3,1,3-isomers. The isomers are represented by analogs 1, 2 and 3, respectively. In addition, the pyridyl analogs 4 (symmetrical) and 5 (unsymmetrical with respect to the THIQ ring systems) are described.

The synthetic protocols to prepare the symmetrical analog 1 is described in Scheme 1. The S_{N}Ar reaction of phenol 6 and fluoride 7 yields the biaryl ether 8 in good yield. Formyl reduction, activation, cyanide displacement followed by hydrolysis provides the diacid 9. Coupling with amine 10 to furnish bis-amide 11. Cyclization to 12 via Bischler Napieralski reaction followed by asymmetric reduction and carbamate protection provides the bis-N-BOC THIQ intermediate which was purified via flash column chromatography. The installment of the N-methyl units is accomplished via N-BOC deprotection and alkylation using formaldehyde to provide 1. A slight modification to this synthetic route was utilized to generate compounds (Table 1): 41, 42, 43, 44, and 45.

The preparation of 2 is described in Scheme 2 and is analogous to the synthetic route utilized to generate 1. The use of 3-hydroxy-4-methoxybenzaldehyde 13 in place of 6 permits the efficient synthesis of the target analog 2. A slight modification to this synthetic route was utilized to generate compound 47 (Table 2).

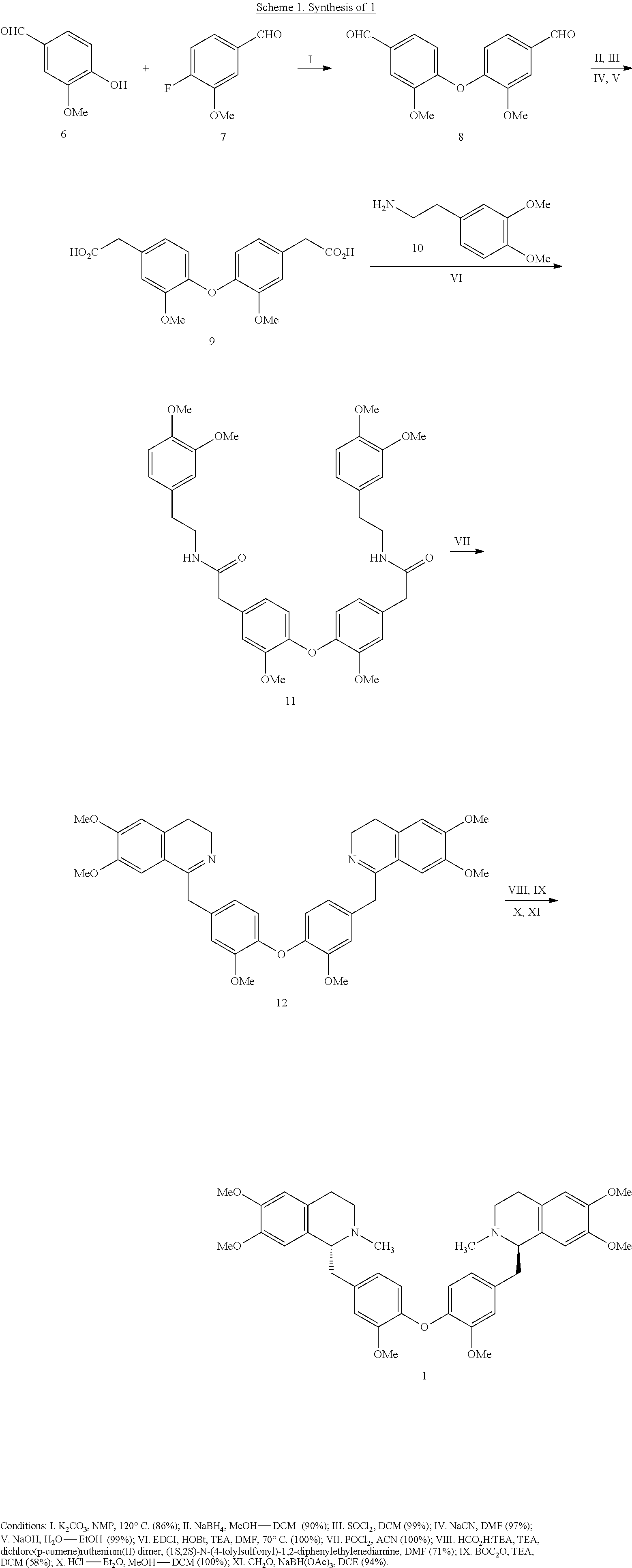

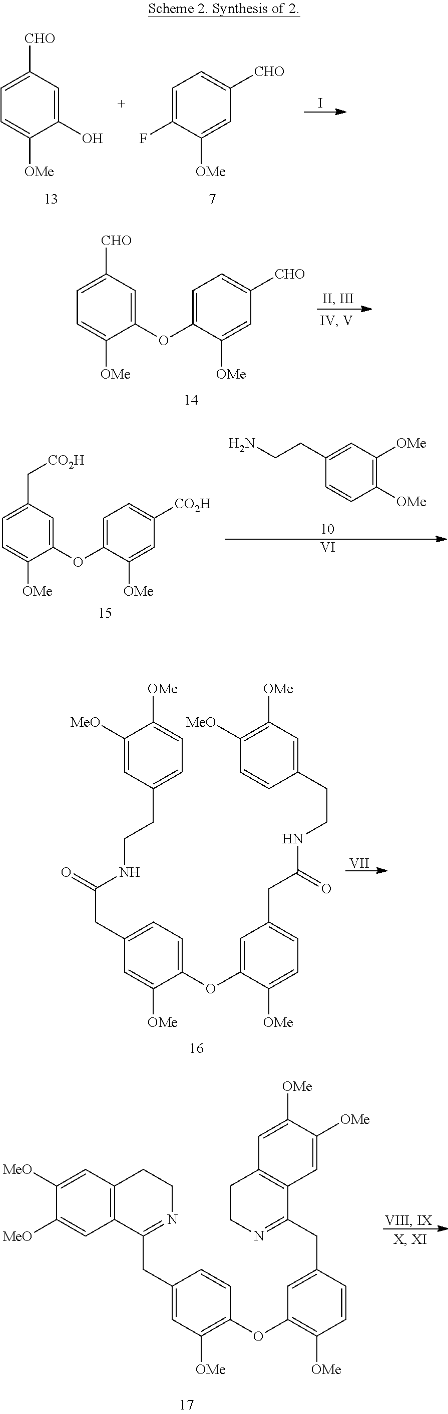

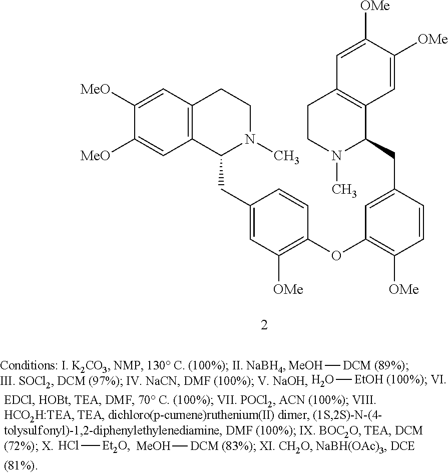

The preparation of 3 is summarized in Scheme 3. This route is analogous to the synthetic routes for 1 and 2 with the exception of the first step which utilizes an Ullmann coupling protocol to generate the bis-aldehyde 19. The remaining steps are identical to the previous routes to generate the key bis-N-BOC intermediate which was directly reduced to 3. A slight modification to this synthetic route was utilized to generate compounds (Table 3): 51 and 52.

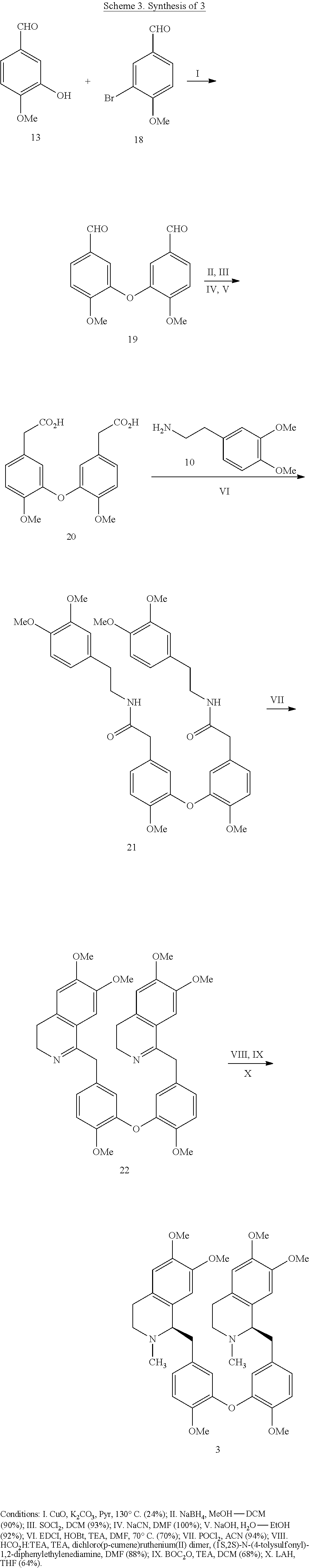

The preparation of compounds 4 is illustrated in Scheme 4. Compound 4 substitutes a pyridine ring to the linking biaryl ether moiety and its synthesis is initiated by treating phenol 6 with 2-broinopyridine 23 to generate 24. The synthetic protocols used to generate 4 are identical to the previously disclosed transformations and are summarized in Scheme 4. A slight modification to this synthetic route was utilized to generate compounds (Tables 1 and 2): 46, 48, 49 and 50.

Compound 5 is an unsymmetrical analog with respect to the two THIQ heterocycles and its preparation is outlined in Scheme 5. The unsymmetrical characteristic of 5 makes it necessary to introduce 29 to generate the arylethyl amide 30. The SN Ar reaction provides the aldehyde 31 which is further converted to the key unsymmetrical bis-amide intermediate 33 as previously described. The remaining synthetic steps to generate 5 have previously been described.

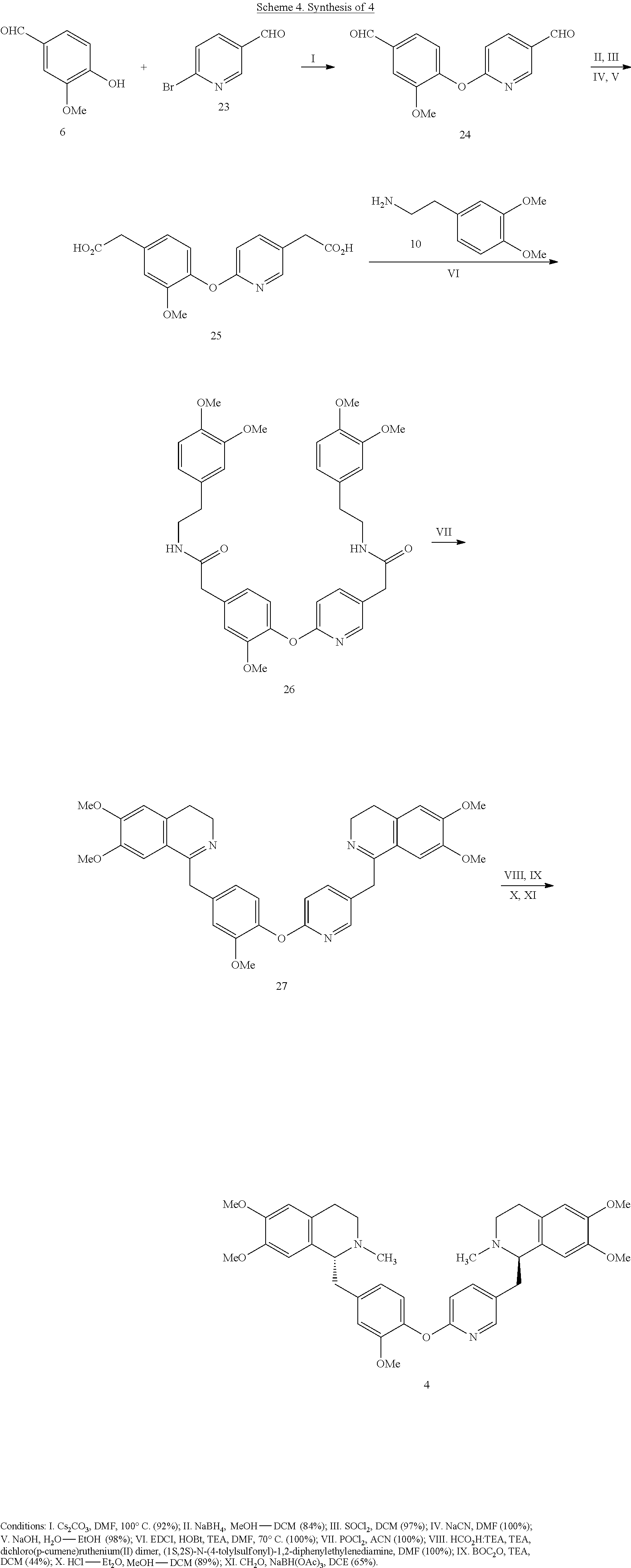

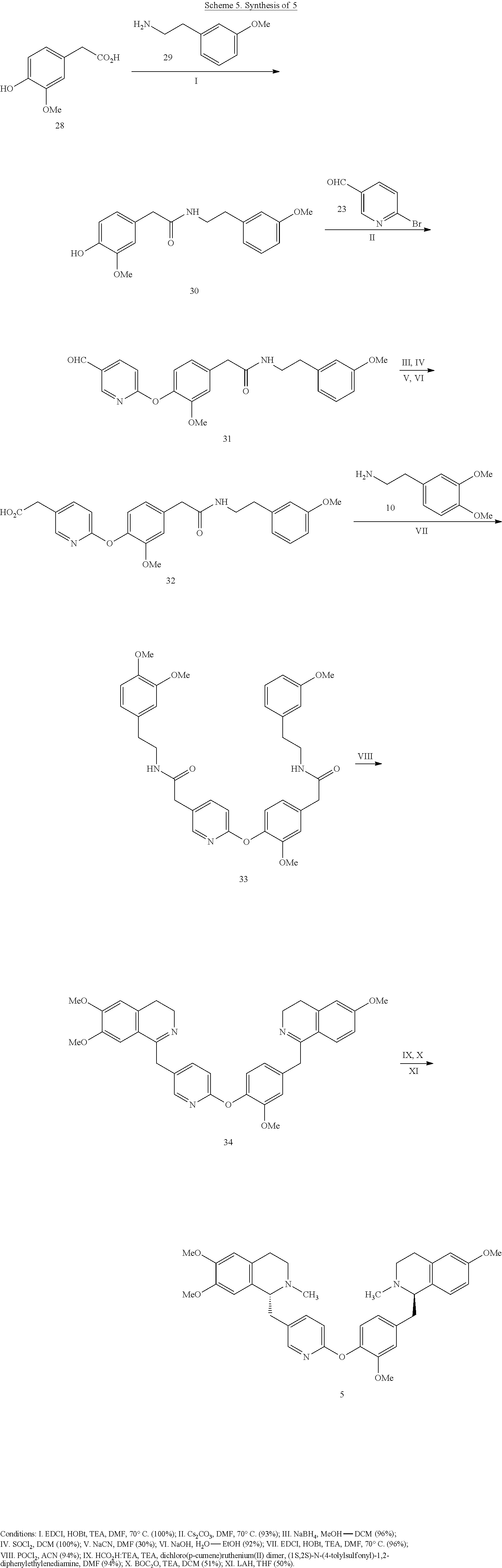

Reference herein is also made to a convergent synthetic route that provides a more structurally diverse set of compounds as summarized in Scheme 6. This strategy targets a variety of phenols (184-10) and arylbromides (IE4-04) which are coupled via an Ullmann protocol to provide the bis-N-formyl intermediate. Hydride reduction produces the target analog B4-14. This synthetic route was utilized to generate the following compounds (Tables 1, 2 and 3): B4-24, B4-17, B4-27, B4-34, B4-37, B4-26, B4-21, B4-22, B4-12, B4-31, B4-32, B4-36, B4-25, B3-34, B3-12 B3-42, B3-46, B3-36 and B3-35.

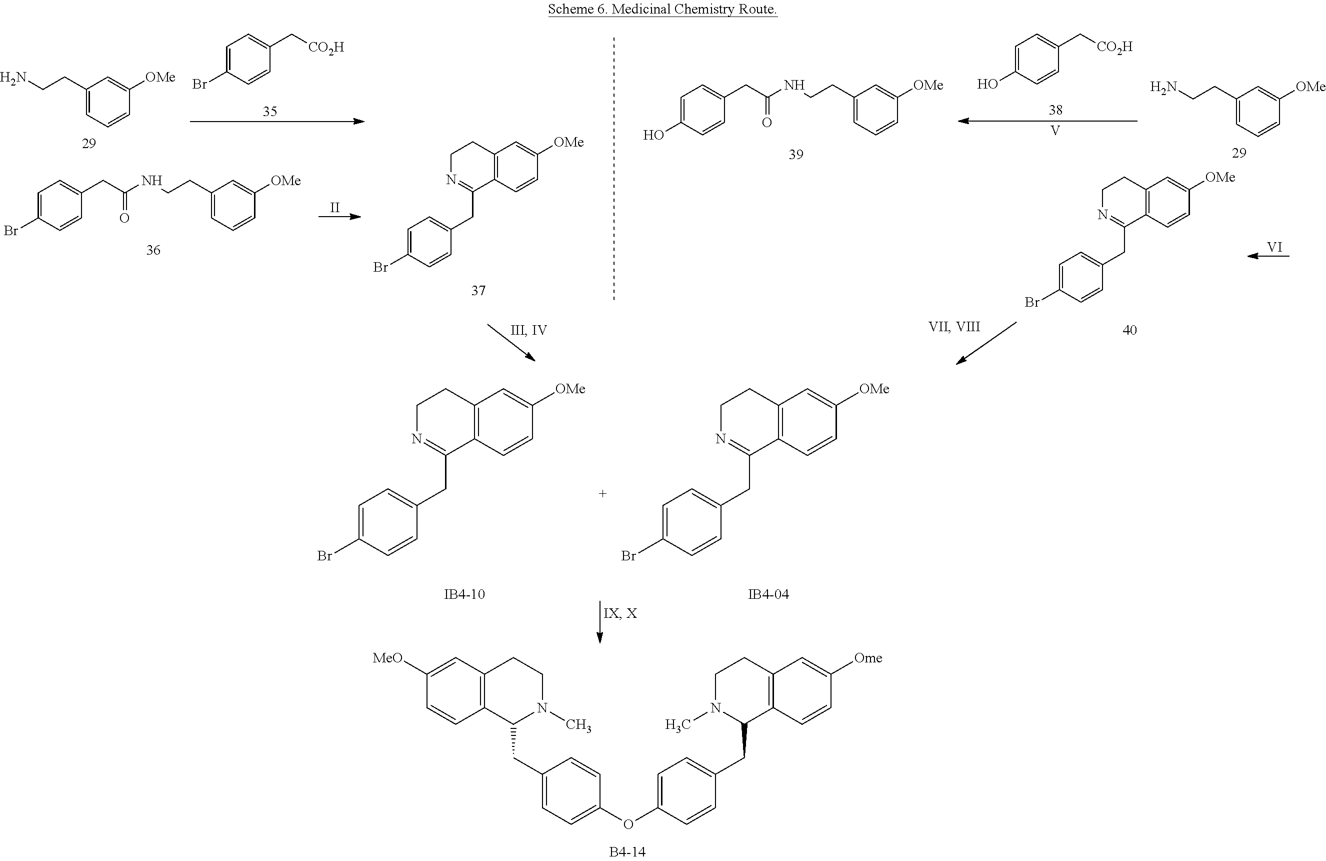

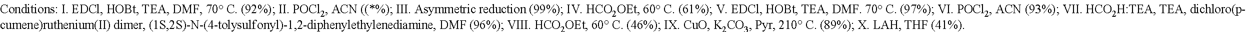

### Structure Activity Relationship (SAR)

The synthetic analogs herein were evaluated for their antiviral activity (Ebola virus, EBOV) and off-target calcium channel activity (L-activity). One preferred outcome was to increase the EBOV activity while diminishing their L-channel activity. The data for the 1,4-1,4-analogs are summarized in Table 1 which show EBOV (IC₅₀/nM) and L-channel (IC₅₀/μM) activities. Table 1 also includes tetrandrine (TETN) as a reference to the synthetic analogs. The parent analog B4-14 shows good activity in both the EBOV and L-channel activity. Interestingly, the introduction of methoxy groups at both the linking biaryl ether and the THIQ heterocycles led to improved EBOV activity and a decrease of L-channel activity (B4-14 vs. 1). The same observation is made in the phenoxypyridine analogs where a dramatic drop in L-channel activity is observed with no significant effect on EBOV activity (5 vs. 4). Lastly, the EBOV activities of these analogs were similar to the natural product TETN.

A similar observation is made for the data of the 1,4,3-analogs as summarized in Table 2. The introduction of methoxy groups at both the linking biaryl ether and the THIQ heterocycle led to improved EBOV activity and a decrease of L-channel activity (B4-21 vs. 2). In this case the phenoxypyridine analog (48) did not show a drop in L-channel activity although it lacks a methoxy group at the linking unit.

Lastly, similar observations exist for the data of the 1,3-1,3-analogs as summarized in Table 3. The introduction of methoxy groups at both the linking biaryl ether and the THIQ heterocycle led to good EBOV activity and decreased of L-channel activity (B3-12 vs. 3). A significant drop in L-channel activity is seen by the presence of a single methoxy on the linking biaryl moiety (51 vs. 3). The 1,3-1,3-analogs were also observed to have diminished EBOV activities relative to TETN.

TABLE 1
1,4-1,4-Analogs

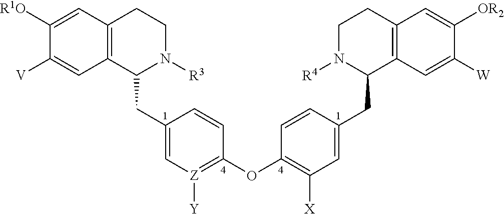

| | | | | | | | | | | | | L- |
|---|---|---|---|---|---|---|---|---|---|---|---|---|
| | | | | | | | | | | EBOV | MARV | Activity |
| Name | R¹ | V | R² | W | X | Y | Z | R³ | R⁴ | IC₅₀ nM | IC₅₀ nM | IC₅₀ μM |
| B4-14^{RR} | Me | H | Me | H | H | H | C | Me | Me | 511 ± 55 | 195 ± 24 | 0.68 ± 0.14 |
| B4-14^{RS} | Me | H | Me | H | H | H | C | Me | Me | 776 ± 68 | 213 ± 88 | 0.52 ± 0.07 |
| B4-14^{SS} | Me | H | Me | H | H | H | C | Me | Me | 706 ± 68 | 284 ± 50 | <0.33 |
| B4-14^{RR} | * | H | Me | H | H | H | C | Me | Me | 235 ± 22 | 108 ± 35 | 0.43 ± 0.13 |
| 41 | Me | H | Me | H | H | H | C | i-Pr | i-Pr | 249 ± 19 | 188 ± 40 | 0.61 ± 0.25 |
| 42 | Me | H | Me | H | H | H | C | Bn | Bn | >1000 | >1000 | 39.44 ± 7.63 |
| 43 | Me | H | Me | H | H | H | C | C(O)CH₂N(Me)₂ | C(O)CH₂N(Me)₂ | 145 ± 24 | 88 ± 23 | <0.33 |
| B4-24 | Me | OMe | Me | H | H | H | C | Me | Me | 125 ± 26 | 130 ± 49 | 1.48 ± 0.22 |
| B4-17 | Me | H | Me | H | H | OMe | C | Me | Me | 134 ± 16 | 133 ± 26 | 0.70 ± 0.22 |
| B4-27 | Me | OMe | Me | H | H | OMe | C | Me | Me | 125 ± 11 | 83 ± 13 | 3.08 ± nd |
| 1 | Me | OMe | Me | OMe | OMe | OMe | C | Me | Me | 730 ± 60 | 506 ± 87 | 16.68 ± 3.37 |
| 44 | Me | OMe | Me | OMe | OMe | OMe | C | cyc-Pr | cyc-Pr | nd | nd | nd |

| | | | | | | | | | | | |
|---|---|---|---|---|---|---|---|---|---|---|---|
| B4-34 | Dioxolane | Me | H | H | H | C | Me | Me | 65 ± 16 | 148 ± 48 | 0.80 ± 0.10 |
| B4-37 | Dioxolane | Me | H | OMe | H | C | Me | Me | 98 ± 23 | 101 ± 31 | 2.24 ± 0.47 |

| | | | | | | | | | | |
|---|---|---|---|---|---|---|---|---|---|---|
| 45 | Dioxolane | Dioxolane | OMe | OMe | C | Me | Me | 372 ± 34 | 339 ± 56 | 2.60 ± 0..51 |

| | | | | | | | | | | | |
|---|---|---|---|---|---|---|---|---|---|---|---|
| 5 | Me | H | Me | OMe | OMe | N | Me | Me | 505 ± 32 | 414 ± 70 | 8.03 ± 0.76 |
| 4 | Me | OMe | Me | OMe | OMe | N | Me | Me | 446 ± 57 | 566 ± 108 | 43.76 ± 11.08 |
| 46 | Me | OMe | Me | OMe | OMe | N | cyc-Pr | cyc-Pr | nd | nd | nd |

| | | | |
|---|---|---|---|
| TETN | 292 ± 31 | 370 ± 95 | 46.80 ± 10.84 |

* Des-OMe analog of B4-14.

TABLE 2
1,4-1,3-Analogs

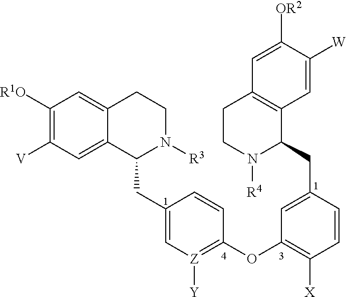

| | | | | | | | | | | | | L- |
|---|---|---|---|---|---|---|---|---|---|---|---|---|
| | | | | | | | | | | EBOV | MARV | Activity |
| Name | R¹ | V | R² | W | X | Y | Z | R³ | R⁴ | IC₅₀ nM | IC₅₀ nM | IC₅₀ μM |
| B4-26 | Me | OMe | Me | OMe | H | H | C | Me | Me | 530 ± 81 | 770 ± 89 | 0.63 ± 0.21 |
| B4-21 | Me | OMe | Me | H | H | H | C | Me | Me | 424 ± 82 | 467 ± 93 | 0.68 ± 0.17 |
| B4-22 | Me | OMe | Me | H | OMe | H | C | Me | Me | 200 ± 30 | 173 ± 36 | 2.27 ± 0.30 |
| B4-12 | Me | H | Me | H | OMe | H | C | Me | Me | 1218 ± 288 | 562 ± 45 | 0.47 ± 0.06 |
| 47 | Me | OMe | Me | OMe | OMe | H | C | Me | Me | nd | nd | nd |
| 2 | Me | OMe | Me | OMe | OMe | OMe | C | Me | Me | 1052 ± 90 | 1235 ± 269 | 27.26 ± 7.90 |

| | | | | | | | | | | | |
|---|---|---|---|---|---|---|---|---|---|---|---|
| B4-31 | Dioxolane | Me | H | H | H | C | Me | Me | 775 ± 156 | 1185 ± 105 | 0.38 ± 0.04 |
| B4-32 | Dioxolane | Me | H | OMe | H | C | Me | Me | 795 ± 210 | 1010 ± 108 | 0.40 ± 0.07 |
| B4-36 | Dioxolane | Me | OMe | H | H | C | Me | Me | 415 ± 43 | 487 ± 88 | 0.95 ± 0.41 |

| | | | | | | | | | | | |
|---|---|---|---|---|---|---|---|---|---|---|---|
| B4-25 | Me | OMe | Dioxolane | H | H | C | Me | Me | 717 ± 89 | 292 ± 27 | 1.72 ± 0.29 |
| B4-25* | Me | OMe | Dioxolane | H | H | C | Me | H | 850 ± 114 | 364 ± 34 | 1.62 ± 0.32 |
| B3-34^{RR} | Me | H | Dioxolane | H | H | C | Me | Me | 887 ± 60 | 530 ± 41 | 0.350 ± 0.05 |
| B3-34^{SS} | Me | H | Dioxolane | H | H | C | Me | Me | 992 ± 139 | 888 ± 95 | 0.73 ± 0.11 |

| | | | | | | | | | | | |
|---|---|---|---|---|---|---|---|---|---|---|---|
| 48 | Me | OMe | Me | OMe | H | N | Me | Me | 875 ± 110 | 1486 ± 320 | 4.01 ± 0.25 |
| 49 | Me | OMe | Me | OMe | OMe | N | Me | Me | nd | nd | nd |
| 50 | Me | OMe | Me | OMe | OMe | N | cyc-Pr | cyc-Pr | nd | nd | nd |

| | | | |
|---|---|---|---|
| TETN | 292 ± 31 | 370 ± 95 | 46.80 ± 10.84 |

* Des-NMe analog of B4-25.

TABLE 3
1,3-1,3-Analogs

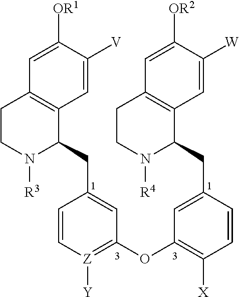

| | | | | | | | | | | EBOV | MARV | L-Activity |
|---|---|---|---|---|---|---|---|---|---|---|---|---|
| Name | R¹ | V | R² | W | X | Y | Z | R³ | R⁴ | IC₅₀ nM | IC₅₀ nM | IC₅₀ μM |
| B3-12 | Me | H | Me | H | H | OMe | C | Me | Me | 1106 ± 179 | 839 ± 158 | 0.54 ± 0.06 |
| B3-42 | Me | H | Me | H | OMe | OMe | C | Me | Me | 617 ± 45 | 418 ± 47 | 0.42 ± 0.07 |
| B3-46 | Me | H | Me | OMe | OMe | H | C | Me | Me | 1094 ± 154 | 282 ± 29 | 1.04 ± 0.41 |
| 51 | Me | OMe | Me | OMe | OMe | H | C | Me | Me | 1091 ± 72 | 1177 ± 179 | 2.81 ± 0.42 |
| 3 | Me | OMe | Me | OMe | OMe | OMe | C | Me | Me | 1222 ± 119 | 1102 ± 222 | 26.7 ± n.d |

| | | | | | | | | | | | |
|---|---|---|---|---|---|---|---|---|---|---|---|
| B3-36^{R,R} | Dioxolane | Me | OMe | H | H | C | Me | Me | 413 ± 51 | 495 ± 66 | 7.74 ± 214 |
| B3-36^{S,R} | Dioxolane | Me | OMe | H | H | C | Me | Me | 967 ± 76 | 743 ± 64 | <0.33 |

| | | | | | | | | | | |
|---|---|---|---|---|---|---|---|---|---|---|
| B3-35 | Dioxolane | Dioxolane | H | H | C | Me | Me | 789 ± 97 | 537 ± 60 | <0.33 |
| 52 | Dioxolane | Dioxolane | OMe | OMe | C | Me | Me | 615 ± 40 | 585 ± 154 | 0.48 ± 0.04 |

| | | | |
|---|---|---|---|
| TETN | 292 ± 31 | 370 ± 95 | 46.8 ± 10.84 |

### Solubility

A turbidimetric solubility assay was conducted by diluting test compound solutions prepared in a 1% DMSO in aqueous buffer (0.01 M phosphate buffered saline pH 7.4). An excitation wavelength of 620 nm is used with 7 replicate readings per well. All candidate analogs identified in FIG. 1 were screened did not show any precipitation across the range of concentrations tested up to a top concentration of 100 μM, as shown in Table 4.

TABLE 4
Solubility Screening

| | Solubility |
|---|---|
| Compound | (μM) |
| 1 | >100 |
| 2 | >100 |
| 3 | >100 |
| 4 | >100 |
| 5 | >100 |
| TETN | >100 |

### Murine In Vivo

A murine study was conducted to evaluate the efficacy of analogs 1-4 in FIG. 1 and TETN. Their efficacy was measured against EBOV virus (1000 pfu virus in 0.2 mL saline) administered via IP route to BALB/c mice at eight to nine weeks of age. Three animals per group of ten were euthanized on day three (prior to virus load peak) and virus loads were determined in serum and liver tissue. During the study, animals showing terminal illness (as determined by clinical scores) were euthanized. Any surviving animals were euthanized at the end of the study to determine viral load in serum and liver tissue.

The study was to evaluate the effectiveness of select compounds against an Ebola virus challenge. Compounds 1 and 2 in FIG. 1 performed positively, having 100% survival in their respective groups as summarized in Table 5. The most effective analogs, compounds 1 and 2 show statistically significant improvement when compared to TETN and saline. In addition, compounds 3 and 4 in FIG. 1 show good efficacy at this dose and comparable to TETN.

TABLE 5
Efficacy Study

| Compound | Dose (mg/kg) | Survival | % Survival |
|---|---|---|---|
| Saline | N/A | 3/7 | 43 |
| 1 | 50 | 7/7 | 100 |
| 2 | 50 | 7/7 | 100 |
| 3 | 50 | 5/7 | 71 |
| 4 | 50 | 5/7 | 71 |
| TETN | 50 | 4/7 | 57 |

Based on the in vivo results, Compound 1 and 2 were selected to proceed into a minimum effective dose in vivo study. Three doses were selected and the results are summarized in Table 6. Overall, the test articles appear to suppress the disease symptoms when compared to the saline control. All control animals succumbed to disease. On the other hand, compounds 1 and 2 in FIG. 1 show good efficacy at 50 and 25 mg/kg. Compound 2 showed moderate efficacy at the low dose of 5 mg/kg.

TABLE 6
Minimum Effective Dose Study

| Compound | Dose (mg/kg) | Survival | % Survival |
|---|---|---|---|
| Saline | N/A | 0/7 | 0 |
| 1 | 50 | 4/7 | 57 |
| 1 | 25 | 4/7 | 57 |
| 1 | 5 | 0/7 | 0 |
| 2 | 50 | 4/7 | 57 |
| 2 | 25 | 4/7 | 57 |
| 2 | 5 | 3/7 | 43 |

### Synthetic Procedures

General procedure for the preparation of phenylacetamides.

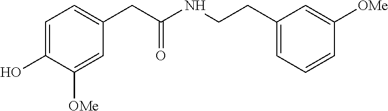 [SMILES: COc1cccc(CCNC(=O)Cc2ccc(O)c(OC)c2)c1]

Preparation of 2-(4-hydroxy-3-methoxyphenyl)-N-[2-(3-methoxyphenyl)ethyl]acetamide. To a suspension of (4-hydroxy-3-methoxyphenyl)acetic acid (6.18 g, 33.93 mmol), 2-(3-methoxyphenyl)ethan-1-amine (5.13 g, 33.93 mmol), HOBt (5.50 g, 40.71 mmol) and TEA (4.12 g, 40.71 mmol) in DMF (68 mL) was added EDCl (7.80 g, 40.71 mmol). The resulting suspension was stirred (70° C, 4 hr). The reaction mixture was cooled (RT) and partitioned with EtOAc (70 mL) and H₂O (70 mL). The aqueous layer was extracted with EtOAc (70 mL). The combined organic layers were washed with 1 M HCl (2×100 mL), saturated NaHCO₃ (2×100 mL), brine (100 mL), dried (Na₂SO₄), filtered and concentrated in vacuo to provide the title compound as an amber oil (9.39 g, 88%): ¹H NMR (CDCl₃, 400 MHz) δ7.14 (t, J=8.6 Hz, 1H), 6.84 (d, J=8.0 Hz, 1H), 6.72-6.75 (m 1H), 6.59-6.65 (m 4H), 5.80 (br s, 1H), 5.46 (br t, 1H), 3.82 (s, 3H), 3.77 (s, 3H), 3.46 (q, J=6.8 Hz, 2H), 3.45 (s, 2H), 2.70 (t, J=6.8 Hz, 2H). ¹³C NMR (CDCl₃, 100 MHz) δ 171.44, 159.78, 146.90, 145.02, 140.23, 129.55, 126.41, 122.28, 120.98, 114.80, 114.40, 111.78, 111.73, 55.88, 55.13, 43.51, 40.52, 35.42 ppm. LCMS m/z (relative intensity) 316.0 [M+1]⁺ (100); 338.0 [M+Na]⁺ (15).

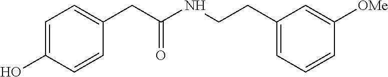 [SMILES: COc1cccc(CCNC(=O)Cc2ccc(O)cc2)c1]

2-(4-Hydroxyphenyl)-N-[2-(3-methoxyphenyl)ethyl]acetamide. Tan solid (9.13 g, 97%): ¹H NMR (DMSO-d₆, 400 MHz) δ9.19 (s, 1H), 7.93 (br t, 1H), 7.12-7.16 (m, 1H), 6.97-6.99 (m, 2H), 6.69-6.74 (m, 3H), 6.63-6.65 (m, 2H), 3.68 (s, 3H), 3.21-3.26 (m, 4H), 2.64 (t, J=7.2 Hz, 2H). ¹³C NMR (DMSO-d₆, 100 MHz) 5171.00, 159.69, 156.28, 141.48, 130.29, 129.69, 126.96, 121.33, 115.38, 114.65, 111.98, 55.27, 42.04, 40.60, 35.57 ppm.

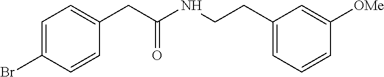 [SMILES: COc1cccc(CCNC(=O)Cc2ccc(Br)cc2)c1]

2-(4-Bromophenyl)-N-[2-(3-methoxyphenyl)ethyl]acetamide. Tan solid (14.9 g, 92%): ¹H NMR (CDCl₃, 400 MHz) δ7.38 (dd, J=6.6, 1.8 Hz, 2H), 7.15-7.11 (m, 2H), 6.99 (d, J=8.4 Hz, 2H), 6.73-6.70 (m, 1H), 6.59-6.56 (m, 2H), 5.43 (br s, 1H), 3.74 (s, 3H), 3.45-3.40 (m, 4H), 2.68 (t, J=6.8 Hz, 2H); LCMS m/z (relative intensity) 348.1 [M+1]⁺ (100), 350.1 (100); 370.1 [M+Na]⁺ (100), 372.0 (100).

General procedure for the preparation of bi-aryl ethers via S_{N}Ar Reaction.

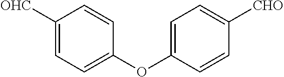 [SMILES: O=Cc1ccc(Oc2ccc(C=O)cc2)cc1]

Preparation of 4,4′-oxydibenzaldehyde. To a solution of 4-hydroxybenzaldehyde (4.00 g, 32.23 mmol) in DMF (65 mL) was added K₂CO₃ (8.90 g, 64.45 mmol) and 4-fluorobenzaldehyde (4.00 g, 32.33 mmol). The resulting suspension was stirred (90° C., 17.5 hr). The reaction mixture was heated further (110° C., 7.5 hr). The reaction mixture was treated with K₂CO₃ (4.50 g) and heated (120° C., 16 hr). The reaction mixture was cooled (RT) and partitioned with EtOAc (100 mL) and H₂O (100 mL). The aqueous layer was extracted with EtOAc (100 mL). The combined organic layers were washed with 1 M HCl (150 mL), saturated NaHCO₃ (150 mL), dried (Na₂SO₄), filtered and concentrated in vacuo to provide the title compound as a tan solid (7.00 g, 96%): ¹H NMR (CDCl₃, 400 MHz) δ9.99 (s, 1H), 7.92-7.95 (m, 2H), 7.17-7.20 (m, 2H). ¹³C NMR (CDCl₃, 100 MHz) δ 190.61, 161.00, 132.59, 132.07, 119.37 ppm. LCMS m/z (relative intensity) 227.0 [M+1]⁺ (100%).

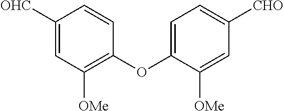 [SMILES: COc1cc(C=O)ccc1Oc1ccc(C=O)cc1OC]

Preparation of 4,4′-oxybis (3-methoxybenzaldehyde). Amber oil (8.63 g, 92%): NMR (CDCl₃, 400 MHz) δ9.93 (s, 1H), 7.55 (d, J=1.6 Hz, 1H), 7.43 (dd, J=8.0, 2.0 Hz, 1H), 6.98 (d, J=8.0 Hz, 1H), 3.94 (s, 3H). ¹³C NMR (CDCl₃, 400 MHz) δ190.86, 151.00, 150.26, 133.25, 125.54, 118.67, 110.85, 56.15 ppm. LCMS m/z (relative intensity) 287.0 [M+1]⁺ (100); 309.0 [M+Na]⁺ (20%).

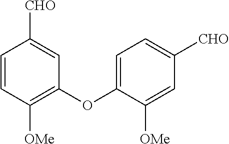 [SMILES: COc1cc(C=O)ccc1Oc1cc(C=O)ccc1OC]

3-(4-Formyl-2-methoxyphenoxy)-4-methoxybenzaldehyde. Amber oil (17.3 g, 100%): ¹H NMR (CDCl₃, 400 MHz) δ9.91 (s, 1H), 9.85 (s, 1H), 7.73 (dd, J=8.4, 2.0 Hz, 1H), 7.54 (dd, J=2.0 Hz, 1H), 7.50 (d, J=2.0 Hz, 1H), 7.38 (dd, J=8.2, 1.8 Hz, 1H), 7.14 (d, J=8.4 Hz, 1H), 6.83 (d, J=8.0 Hz, 1H), 3.96 (s, 3H), 3.93 (s, 2H). ¹³C NMR (CDCl₃, 100 MHz) δ190.86, 190.10, 155.11, 151.36, 150.55, 144.81, 132.57, 130.28, 129.04, 125.65, 120.15, 116.87, 112.24, 110.76, 56.31, 56.18 ppm. LCMS m/z (relative intensity) 287.0 [M+1]⁺ (100%); 309.0 [M+Na]⁺ (20%).

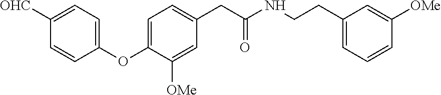 [SMILES: COc1cccc(CCNC(=O)Cc2ccc(Oc3ccc(C=O)cc3)c(OC)c2)c1]

Amide. Tan Solid (3.11 g, 53%): ¹H NMR (CDCl₃, 400 MHz) δ9.90 (s, 1H), 7.83 (dd, J=6.8, 2.0 Hz, 2H), 7.18 (t, J=8.1 Hz, 1H), 7.02 (d, J=8.1 Hz, 1H), 6.97 (dd, J=6.8, 2.0 Hz, 2H), 6.87 (d, J=2.4 Hz, 1H), 6.73-6.80 (m, 2H), 6,66-6.67 (m, 2H), 5.53 (br s, 1H), 3.77 (s, 3H), 3.75 (s, 3H), 3.50-3.55 (m, 4H), 2.76 (t, J=6.8 Hz, 2H). LCMS m/z (relative intensity) 420.0 [M+1]⁺ (100); 442.0 [M+Na]⁺ (10).

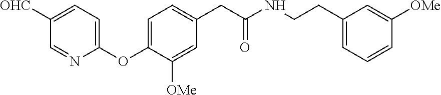 [SMILES: COc1cccc(CCNC(=O)Cc2ccc(Oc3ccc(C=O)cn3)c(OC)c2)c1]

Amide. Tan semisolid (1.44 g, 28%): NMR (CDCl₃, 400 MHz) δ9.96 (d, J=0.4 Hz, 1H), 8.56 (dd, J=2.4, 0.8 Hz, 1H), 8.17 (dd, J=8.4, 2.4 Hz, 1H), 7.18 (t, J=8.0 Hz, 1H), 7.05-7.10 (m, 2H), 6.87 (d, J=1.6 Hz, 1H), 6.80-6.83 (m, 1H), 6.74-6.76 (m, 1H), 6.65-6.67 (m, 2H), 5.55 (br t, 1H), 3.78 (s, 3H), 3.71 (s, 3H), 3.48-3.54 (m, 4H), 2.76 (t, J=6.8 Hz, 2H). LCMS m/z (relative intensity) 421.0 [M+1]⁺ (100); 443.0 [M+Na]⁺ (10).

General procedure for the preparation of bi-aryl ethers via Ullmann Coupling.

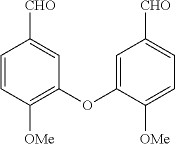 [SMILES: COc1ccc(C=O)cc1Oc1cc(C=O)ccc1OC]

Preparation of 3,3′-oxybis(4-methoxybenzaldehyde). To a suspension of 3-hydroxy-4-methoxybenzaldehyde (11.30 g, 52.58 mmol), 3-bromo-4-methoxybenzaldehyde (4.00 g, 26.29 mmol) and CuO (2.09 g, 26.29 mmol) in pyridine (44 mL) was added K₂CO₃ (3.64 g, 26.29 mmol). The resulting suspension was stirred (130° C., 43 hr). The reaction mixture was cooled (RT) filtered (Celite), the solid was washed with EtOAc (100 mL) and acetone (100 mL). The filtrate was concentrated in vacuo and the resulting residue was partitioned with EtOAc (200 mL) and 1 M HCl (150 mL). The aqueous layer was extracted with EtOAc (200 mL). The combined organic layers were washed with 1 M HCl (2×200 mL), dried (Na₂SO₄), filtered and concentrated in vacuo. The resulting residue was purified by flash column chromatography (SiO₂) using EtOAc:hexanes (10-75%) to provide the title compound as a tan solid (1.8 g, 24%): ¹H NMR (CDCl₃, 400 MHz) δ9.82 (s, 1H), 7.67 (dd, J=8.4, 2.0 Hz, 1H), 7.35 (d, J=2.0 Hz, 1H), 7.12 (d, J=8.4 Hz, 1H), 3.96 (s, 3H). LCMS m/z (relative intensity) 287.0 [M+1]⁺ (100); 309.0 [M+Na]⁺ (20).

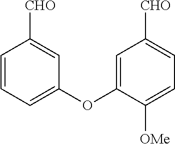 [SMILES: COc1ccc(C=O)cc1Oc1cccc(C=O)c1]

3-(3-Formylphenoxy)-4-methoxybenzaldehyde. Brown residue (1.07 g, 8%): ‘H NMR (CDCl₃, 400 MHz) δ9.56 (s, 1H), 9.86 (s, 1H), 7.74 (dd, J=8.4, 2.0 Hz, 1H), 7.60-7.62 (m, 1H), 7.54 (d, J=2.0 Hz, 1H), 7.51 (t, J=7.8 Hz, 1H), 7.36-7.38 (m, 1H), 7.25-7.28 (m, 1H), 7.14 (d, J=8.4 Hz, 1H), 3.92 (s, 3H). ¹³C NMR (CDCl₃, 100 MHz) δ191.59, 190.18, 158.10, 156.64, 144.92, 138.04, 130.48, 130.38, 129.19, 125.18, 123.60, 120.99, 116.58, 112.43, 56.28 ppm. LCMS m/z (relative intensity) 257.0 [M+1]⁺ (100); 276.0 [M+Na]⁺ (15).

General procedure for the preparation of bi-aryl ethers via S_{N}Ar Reaction.

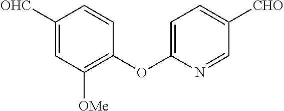 [SMILES: COc1cc(C=O)ccc1Oc1ccc(C=O)cn1]

Preparation of 6-(4-formyl-2-methoxyphenoxy)pyridine-3-carbaldehyde. To a suspension of 4-hydroxy-3-methoxybenzaldehyde (5.00 g, 32.86 mmol) and 6-bromopyridine-3-carbaldehyde (6.11 g, 32.86 mmol) in DMF (66 mL) was added Cs₂CO₃ (21.41 g, 65.72 mmol). The resulting suspension was stirred (100° C., 2.5 hr). The reaction mixture was cooled (RT) and partitioned with EtOAc (100 mL) and H₂O (100 mL). The aqueous layer was extracted with EtOAc (100 L). The combined organic layers were washed with 1 M HCl (2×100 mL), saturated NaHCO₃ (2×100 mL), dried (Na₂SO₄), filtered and concentrated in vacuo to provide the title compound as a tan solid (7.06 g, 89%): NMR (CDCl₃, 400 MHz) δ9.99 (s, 2H), 8.56 (dd, J=2.4, 0.8 Hz, 1H), 8.22 (dd, J=8.4, 2.4 Hz, 1H), 7.55-7.57 (m, 2H), 7.35 (d, J=8.4 Hz, 1H), 7.13-7.15 (m, 1H), 3.83 (s, 3H). LCMS m/z (relative intensity) 257.9 [M+1]⁺ (100); 276.0 [M+Na]⁺ (20).

General procedure for the preparation of diols.

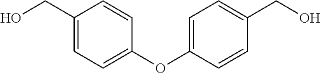 [SMILES: OCc1ccc(Oc2ccc(CO)cc2)cc1]

Preparation of (oxydibenzene-4,1-diyl)dimethanol. To a solution of 4,4′-oxydibenzaldehyde (6.20 g, 27.41 mmol) in MeOH:DCM (55 mL) was added NaBH₄ (2.28 g, 60.29 mmol). The resulting suspension was stirred (RT, 1.0 hr). The reaction mixture was concentrated in vacuo and the resulting residue was suspended in H₂O (100 mL). The reaction mixture was filtered and the resulting solid was dried under high vacuum to provide the title compound as a white solid (5.13 g, 81%): NMR (DMSO-d₆, 400 MFIz) δ7.27 (dd, J=6.5, 2.1 Hz, 4H), 6.90 (dd, J=6.5, 2.1 Hz, 4H), 4.42 (s, 4H). ¹³C NMR (CDCl₃, 100 MHz) δ156.05, 137.96, 128.61, 118.62, 62.83 ppm. LCMS m/z (relative intensity) 413.0 [M−OH]⁺ (100).

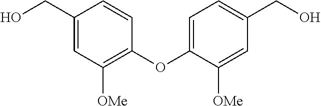 [SMILES: COc1cc(CO)ccc1Oc1ccc(CO)cc1OC]

[Oxybis(3-methoxy-4,1-phenylene)]dimethanol. Amber oil (14.0 g, 99%): ¹H NMR (CDCl₃, 400 MHz) δ7.02 (d, J=1.8 Hz, 1H), 6.83 (dd, J=8.0, 1.8 Hz, 1H), 6.77 (d, J=8.0 Hz, 1H), 4.66 (s, 2H), 3.87 (s, 3H), 1.82 (br s, 1H). ¹³C NMR (CDCl₃, 100 MHz) δ150.51, 145.30, 136.69, 119.27, 118.57, 111.29, 65.20, 55.94 ppm. LCMS m/z (relative intensity) 313.0 [M+Na]⁺ (55); 273.0 [M−OH]⁺ (100).

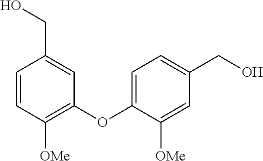 [SMILES: COc1cc(CO)ccc1Oc1cc(CO)ccc1OC]

{4-[5-(Hydroxymethyl)-2-methoxyphenoxy]-3-methoxyphenyl}methanol. Amber oil (15.55 g, 89%): ¹H NMR (CDCl₃, 400 MHz) δ7.03 (dd, J=8.0, 2.0 Hz, 1H), 7.01 (d, J=2.0 Hz, 1H), 6.95 (d, J=8.4 Hz, 1H), 6.79-6.82 (m, 3H), 4.64 (s, 2H), 4.51 (s, 2H), 3.86 (s, 3H), 3.85 (s, 3H). ¹³C NMR (CDCl₃, 100 MHz) δ 150.71, 149.88, 146.18, 145.02, 136.98, 133.72, 122.39, 119.36, 119.13, 117.30, 112.40, 111.49, 65.15, 64.82, 56.10, 55.99 ppm. LCMS m/z (relative intensity) 273.0 [M−OH]⁺ (100); 313.0 [M+Na]⁺ (30).

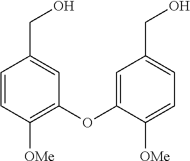 [SMILES: COc1ccc(CO)cc1Oc1cc(CO)ccc1OC]

[Oxybis(4-methoxybenzene-3,1-diyl)]dimethanol. Brown residue (1.41 g, 77%): ¹H NMR (CDCl₃, 400 MHz) δ7.05 (dd, J=8.0, 2.0 Hz, 1H), 6.95 (d, J=8.0 Hz, 1H), 6.83 (d, J=2.0 Hz, 1H), 4.52 (s, 2H), 3.86 (s, 3H). LCMS m/z (relative intensity) 272.9 [M−OH]⁺ (100); 313.0 [M+Na]⁺ (40).

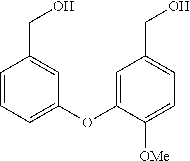 [SMILES: COc1ccc(CO)cc1Oc1cccc(CO)c1]

{3-[5-(Hydroxymethyl)-2-methoxyphenoxy]phenyl}methanol. Amber residue (0.86 g, 79%): ¹H NMR (CDCl₃, 400 MHz) δ7.23 (d, J=8.0 Hz, 1H), 7.06 (dd, J=8.4, 2.0 Hz, 1H), 6.91-6.95 (m, 4H), 6.85-6.88 (m, 1H), 4.55 (s, 2H), 4.49 (s, 2H), 3.81 (s, 3H). ¹³C NMR (CDCl₃, 100 MHz) δ157.96, 150.76, 145.00, 142.77, 133.99, 129.69, 123.48, 121.11, 119.89, 116.61, 115.64, 112.76, 64.78, 64.49, 56.11 ppm. LCMS m/z (relative intensity) 243.0 [M—OH]⁺ (100); 283.1 [M+Na]⁺ (15).

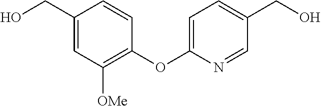 [SMILES: COc1cc(CO)ccc1Oc1ccc(CO)cn1]

{6-[4-(Hydroxymethyl)-2-methoxyphenoxy]pyridine-3-yl}methanol. Tan Solid (4.75 g, 94%): ¹H NMR (CDCl₃, 400 MHz) δ8.04 (d, J=2.4 Hz, 1H), 7.69 (dd, J=8.4, 2.4 Hz, 1H), 7.08 (d, J=8.0 Hz, 1H), 7.05 (d, J=2.0 Hz, 1H), 6.91-6.95 (m, 2H), 4.67 (d, J=5.2 Hz, 2H), 4.60 (d, J=4.8 Hz, 2H), 3.76 (s, 3H). LCMS m/z (relative intensity) 262.0 [M+1]⁺ (100); 284.0 [M+Na]⁺ (10).

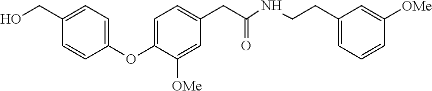 [SMILES: COc1cccc(CCNC(=O)Cc2ccc(Oc3ccc(CO)cc3)c(OC)c2)c1]

Alcohol. Tan Solid (2.07 g, 98%): ¹H NMR (CDCl₃, 400 MHz) δ7.31 (d, J=8.8 Hz, 2H), 7.17 (t, J=8.1 Hz, 1H), 6.92 (d, J=8.8 Hz, 2H), 6.87 (d, J=8.1 Hz, 1H), 6.81 (d, J=2.0 Hz, 1H), 6.73-6.76 (m, 1.H), 6.70 (dd, J=8.1, 2.0 Hz, 1H), 6.63-6.65 (m, 2H), 5.42 (br t, 1H), 4.65 (s, 2H), 3.79 (s, 3H), 3.77 (s, 3H), 3.45-3.52 (m, 4H), 2.74 (t, J=6.8 Hz, 2H). LCMS m/z (relative intensity) 422.0 [M+1]⁺ (100); 444.0 [M+Na]⁺ (10).

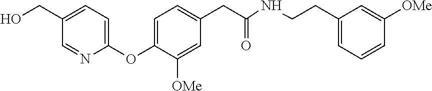 [SMILES: COc1cccc(CCNC(=O)Cc2ccc(Oc3ccc(CO)cn3)c(OC)c2)c1]

Alcohol. Clear semisolid (1.35 g, 93%): NMR (CDCl₃, 400 MHz) δ8.01-8.02 (m, 1H), 7.70 (dd, J=8.4, 2.4 Hz, 1H), 7.17 (t, J=7.6 Hz, 1 H.), 7.04 (d, J=8.0 Hz, 1H), 6.91 (d, J=8.4 Hz, 1H), 6.81 (d, J=1.6 Hz, 1H), 6.72-6.78 (m, 2H), 6.64-6.66 (m, 2H), 5.60 (br t, 1H), 3.77 (s, 3H), 3.70 (s, 3H), 3.52 (s, 2H), 3.47 (q, J=6.8 Hz, 2H), 2.74 (t, J=6.8 Hz, 2H). ¹³C NMR (CDCl₃, 100 MHz) δ170.88, 163.24, 151.96, 159.79, 146.16, 141.75, 140.31, 139.02, 132.59, 130.73, 129.61, 123.34, 121.98, 121.05, 114.46, 113.94, 111.77, 110.71, 62.23, 55.89, 55.14, 43.74, 40.70, 35.45 ppm. LCMS m/z (relative intensity) 423.0 [M+1]⁺ (100).

General procedure for the preparation of di-chlorides

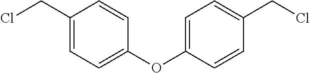 [SMILES: ClCc1ccc(Oc2ccc(CCl)cc2)cc1]

Preparation of bis[4-(chloromethyl)phenyl] ether. To a suspension of (oxydibenzene-4,1-diyl)dimethanol (0.58 g, 2.52 mmol) in DCM (5 mL) was added SOCl₂. (1.20 g, 10.08 mmol, 0.73 mL). The resulting suspension was stirred (RT, 4.5 hr). The reaction mixture was concentrated in vacuo and the resulting residue was partitioned with EtOAc (75 mL) and NaHCO₃ (75 mL).

The organic layer was washed saturated NaHCO₃ (100 mL), dried (Na₂SO₄), filtered and concentrated in vacuo to provide the title compound as a white solid (0.58 g, 86%): NMR (CDCl₃, 400 MHz) δ7.35-7.38 (m, 2H), 6.98-7.01 (m, 2H), 4.58-4.60 (m, 2H). ¹³C NMR (CDCl₃, 100 MHz) δ157.03, 132.62, 130.29, 119.10, 45.84 ppm.

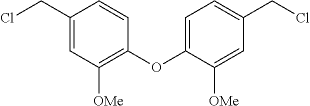 [SMILES: COc1cc(CCl)ccc1Oc1ccc(CCl)cc1OC]

1,1′-Oxybis[4-(chloromethyl)-2-methoxybenzene]. Tan solid (3.01 g, 99%): ¹H NMR (CDCl₃, 400 MHz) δ7.01 (d, J=2.0 Hz, 1H), 6.87 (dd, J=8.0, 2.0 Hz, 1H), 6.77 (d, J=8.0, 1H), 4.58 (s, 2H), 3.87 (s, 3H). LCMS m/z (relative intensity) 291.0 [M−Cl]⁺ (100), 293.1 [M−Cl+2]⁺ (35); 349.0 [M+Na]⁺ (100), 351.0 [M+Na+2]⁺ (60).

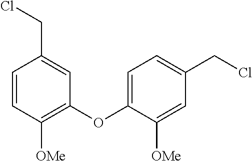 [SMILES: COc1cc(CCl)ccc1Oc1cc(CCl)ccc1OC]

4-(Chloromethyl)-1-[5-(chloromethyl)-2-methoxyphenoxy]-2-methoxybenzene. Amber oil (17.08 g, 97%): ¹H NMR (CDCl₃, 400 MHz) δ7.10 (dd, J=8.0, 2.0 Hz, 1H), 7.02 (d, J=2.0 Hz, 1H), 6.94 (d, J=8.0 Hz, 1H), 6.86-6.89 (m, 2H), 6.77 (d, J=8.0 Hz, 1H), 4.58 (s, 2H), 4.48 (s, 2H), 3.88 (s, 3H), 3.85 (s, 3H). ¹³C NMR (CDCl₃, 100 MHz) δ 150.67, 150.45, 145.86, 145.50, 133.21, 130.21, 124.50, 121.10, 119.37, 118.45, 112.87, 112.42, 56.05, 56.02, 46.34, 46.00 ppm. LCMS m/z (relative intensity) 291.0 [M−Cl]⁺ (100), 293.0 [M−Cl+2]⁺ (35); 349.0 [M+Na]⁺ (70), 351.0 [M+Na+2]⁺ (50).

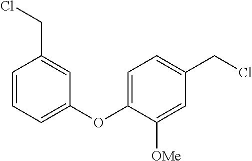 [SMILES: COc1cc(CCl)ccc1Oc1cccc(CCl)c1]

4-(Chloromethyl)-1-[3-(chloromethyl)phenoxy]-2-methoxybenzene. Tan solid (3.40 g, 96%): NMR (CDCl₃, 400 MHz) δ7.24-7.28 (m, 1H), 7.04-7.07 (m, 2H), 6.93-6.97 (m, 3H), 7.87 (dd, J=8.4, 2.4 Hz, 1H), 4.58 (s, 2H), 4.51 (s, 2H), 3.82 (s, 3H). ¹³C NMR (CDCl₃, 100 MHz) δ 157.84, 151.43, 144.74, 139.12, 134.34, 129.88, 122.79, 121.30, 120.99, 117.28, 117.09, 113.14, 55.99, 46.17, 45.82 ppm.

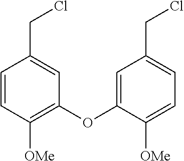 [SMILES: COc1ccc(CCl)cc1Oc1cc(CCl)ccc1OC]

1,1′-Oxybis(5-(chloromethyl)-2-methoxybenzene]. Brown residue (1.5 g, 94%): ¹H NMR (CDCl₃, 400 MHz) δ7.11 (dd, J=8.3, 2.1 Hz, 1H), 6.95 (d, J=8.3 Hz, 1H), 6.87 (d, J=2.1 Hz, 1H), 4.49 (s, 2H), 3.87 (s, 3H).

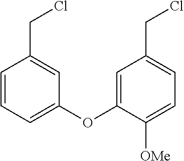 [SMILES: COc1ccc(CCl)cc1Oc1cccc(CCl)c1]

4-(Chloromethyl)-2-[3-(chloromethyl)phenoxy]-1-methoxybenzene. Amber oil (0.82 g, 84%): ¹H NMR (CDCl₃, 400 MHz) δ7.27 (t, J=8.0 Hz, 1H), 7.17 (dd, J=8.2, 2.2 Hz, 1H), 7.06-7.09 (m, 1H), 7.03 (d, J=2.2 Hz, 1H), 6.96-6.98 (m, 2H), 6.86-6.89 (m, 1H), 4.52 (s, 2H), 4.51 (s, 2H), 3.82 (s, 3H). ¹³C NMR (CDCl₃, 100 MHz) δ157.89, 151.55, 144.60, 139.16, 130.53, 129.91, 129.40, 122.78, 121.58, 117.27, 117.02, 112.80, 56.08, 45.86, 45.81 ppm.

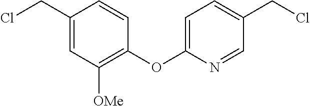 [SMILES: COc1cc(CCl)ccc1Oc1ccc(CCl)cn1]

5-(Chloromethyl)-2-[4-(chloromethyl)-2-methoxyphenoxy]pyridine. Tan Solid (4.73 g, 97%): ¹H NMR (CDCl₃, 400 MHz) δ8.10 (d, J=2.4 Hz, 1H), 7.72 (dd, J=8.6, 2.5 Hz, 1H), 7.10 (d, J=8.0 Hz, 1H), 7.05 (d, J=2.0 Hz, 1H), 7.00 (dd, J=8.0, 2.0 Hz, 1H), 6.95 (d, J=8.5 Hz, 1H), 4.61 (s, 2H), 4.55 (s, 2H), 3.78 (s, 3H). LCMS m/z (relative intensity) 297.9 [M+1]⁺ (100); 299.9 [M+2]⁺ (70).

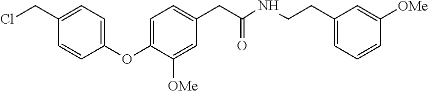 [SMILES: COc1cccc(CCNC(=O)Cc2ccc(Oc3ccc(CCl)cc3)c(OC)c2)c1]

Chloride. Amber oil (2.07 g, 96%): ¹H NMR (CDCl₃, 400 MHz) δ7.31 (d, J=9.2 Hz, 2H), 7.17 (t, J=8.0 Hz, 1H), 6.88-6.92 (m, 3H), 6.82 (d, J=2.0 Hz, 1H), 6.71-6.76 (m, 2H), 6.64-6.66 (m, 2H), 5.48 (br t, 1H), 4.58 (s, 2H), 3.78 (s, 3H), 3.76 (s, 3H), 3.48-3.53 (m, 4H), 2.74 (t, J=6.8 Hz, 2H). LCMS m/z (relative intensity) 440.0 [M+1]⁺ (100); 462.0 [M+Na]⁺ (10).

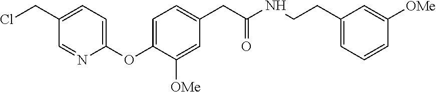 [SMILES: COc1cccc(CCNC(=O)Cc2ccc(Oc3ccc(CCl)cn3)c(OC)c2)c1]

Chloride. White semisolid (1.35 g, 96%): ¹H NMR (CDCl₃, 400 MHz) δ8.09-8.10 (m, 1H), 7.73 (dd, J=8.4, 2.4 Hz, 1H), 7.18 (t, J=8.0 Hz, 1H), 7.06 (d, J=8.0 Hz, 1H), 6.94 (d, J=8.4 Hz, 1H), 6.83 (d, J=2.0 Hz, 1H), 6.78 (dd, J=7.8, 2.2 Hz, 1H), 6.73-6.76 (m, 1H), 6.64-6.67 (m, 2H), 5.54 (br t, 1H), 4.55 (s, 2H), 3.78 (s, 3H), 3.73 (s, 3H), 3.53 (s, 2H), 3.49 (q, J=6.7 Hz, 2H), 2.74 (t, J=6.7 Hz, 2H). ¹³C NMR (CDCl₃, 100 MHz) δ170.70, 163.56, 159.82, 151.95, 147.20, 141.52, 140.29, 139.93, 132.83, 129.64, 127.67, 123.40, 121.93, 121.04, 114.47, 113.88, 111.77, 1.10.97, 55.91, 55.15, 43.81, 43.02, 40.66, 35.47 ppm. LCMS m/z (relative intensity) 441.0 [M+1]⁺ (100), 443.0 [M+2] (50); 463.0 [M+Na]⁺ (10).

General procedure for the preparation of diacetonitriles.

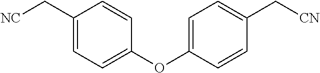 [SMILES: N#CCc1ccc(Oc2ccc(CC#N)cc2)cc1]

Preparation of 2,2′-(oxydibenzene-4 1-diyl)diacetonitrile. To a solution of his[4-chloromethyl)phenyl] ether (0.56 g, 2.10 mmol) in DMF (4 mL) was added NaCN (0.62 g, 12.58 mmol). The resulting suspension was stirred (70° C., 4.5 hr). The reaction mixture was partitioned with EtOAc (50 mL) and H₂O (50 mL). The aqueous layer was extracted with EtOAc (50 mL). The combined organic layers were washed with 1 M HCl (2×100 mL), saturated NaHCO₃ (2×100 mL), dried (Na₂SO₄), filtered and concentrated in vacuo to provide the title compound as a tan solid (0.52 g, 100%): ¹H NMR (CDCl₃, 400 MHz) δ7.30-7.32 (m, 4H), 6.99-7.02 (m, 4H), 3.74 (s, 4H). ¹³C NMR (CDCl₃, 100 MHz) δ156.77, 129.53, 124.96, 119.50, 117.86, 23.00 ppm.

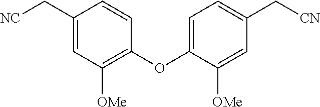 [SMILES: COc1cc(CC#N)ccc1Oc1ccc(CC#N)cc1OC]

2,2′-[Oxybis(3-methoxy-4,1-phenylene)]diacetonitrile. Tan solid (2.75 g, 97%): ¹H NMR (CDCl₃, 400 MHz) δ6.93 (d, J=1.6 Hz, 1H), 6.78-6.83 (m, 2H), 3.88 (s, 3H), 3.74 (s, 2H). LCMS m/z (relative intensity) 331.0 [M+Na]⁺ (100).

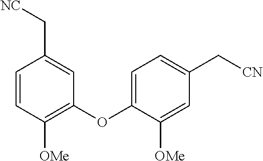 [SMILES: COc1cc(CC#N)ccc1Oc1cc(CC#N)ccc1OC]

{4-[5-(Cyanomethyl)-2-methoxyphenoxy]-3-methoxyphnyl}acetonitrile. Amber oil (16.42 g, 100%): ¹H NMR (CDCl₃, 400 MHz) δ7.04-7.06 (m, 1H), 6.96 (d, J=8.0 Hz, 1H), 6.94 (d, J=1.6 Hz, 1H), 6.82-6.85 (m, 2H), 6.73 (d, J=2.0 Hz, 1H), 3.87 (s, 3H), 3.86 (s, 3H), 3.75 (s, 2H), 3.62 (s, 2H). ¹³C NMR (CDCl₃, 100 MHz) δ150.87, 150.17, 146.04, 145.16, 125.92, 123.48, 122.35, 120.42, 119.38, 118.12, 117.90, 112.89, 112.27, 56.10, 23.38, 22.85 ppm, the remaining two peaks were not detected arid are believed to overlap with the peaks at 118.12 and 56.10 ppm. LCMS m/z (relative intensity) 309.0 [M+1]⁺ (100); 331.0 [M+Na]⁺ (100).

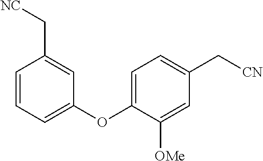 [SMILES: COc1cc(CC#N)ccc1Oc1cccc(CC#N)c1]

{3-[4-(Cyanomethyl)-2-methoxyphenoxy]phenyl}acetonitrile. Amber oil (3.10 g, 100%): ¹H NMR (CDCl3, 400 MHz) δ7.29 (t, J=8.0 Hz, 1H), 7.00-7.03 (m, 1H), 6.97 (br s, 1H), 6.84-6.91 (m, 4H), 3.83 (s, 3H), 3.76 (s, 2H), 3.70 (s, 2H). ¹³C NMR (CDCl3, 100 MHz) δ158.31, 151.87, 144.00, 131.62, 130.34, 127.12, 122.08, 121.90, 120.68, 117.85, 117.65, 116.44, 116.38, 112.58, 56.07, 23.45, 23.41 ppm. LCMS m/z (relative intensity) 279.0 [M+1]⁺ (30), 252.0 [M−CN]⁺ (60); 300.9 [M+Na]⁺ (100).

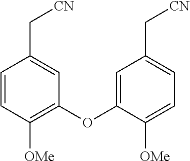 [SMILES: COc1ccc(CC#N)cc1Oc1cc(CC#N)ccc1OC]

2,2′-[Oxybis(4-methoxybenzene-3,1-diyl)]diacetonitrile. Tan solid (1.11 g, 79%): ¹H NMR (CDCl₃, 400 MHz) δ7.05-7.08 (m, 1H), 6.97 (d, J=8.4 Hz, 1H), 6.75 (d, J=2.1 Hz, 1H), 3.87 (s, 3H), 3.63 (s, 2H). LCMS m/z (relative intensity) 309.0 [M+1]⁺ (25); 331.0 [M+Na]⁺ (100).

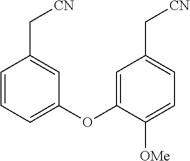 [SMILES: COc1ccc(CC#N)cc1Oc1cccc(CC#N)c1]

{3-[5-(Cyanomethyl)-2-methoxyphenoxy]phenyl}acetonitrile. Amber oil (0.82 g, 84%): ¹H NMR (CDCl₃, 400 MHz) δ7.30 (t, J=8.0 Hz, 1H), 7.14 (dd, J=8.4, 2.4 Hz, 1H), 7.00-7.04 (m, 2H), 6.94 (d, J=2.4 Hz, 1H), 6.84-6.89 (m, 2H), 3.82 (s, 3H), 3.71 (s, 2H), 3.67 (s, 2H). ¹³C NMR (CDCl₃, 100 MHz) δ158.20, 151.29, 144.56, 131.64, 130.38, 124.83, 122.70, 122.19, 121.10, 117.82, 117.61, 116.53, 116.45, 113.33, 56.09, 23.48, 22.78 ppm. LCMS m/z (relative intensity) 279.0 [M+1]⁺ (20), 252.0 [M−CN]⁺ (80); 301.0 [M+Na]⁺ (100).

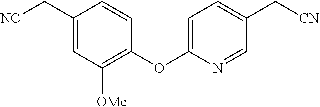 [SMILES: COc1cc(CC#N)ccc1Oc1ccc(CC#N)cn1]

{6-[4-(Cyanomethyl)-2-methoxyphenoxy]pyridine-3-yl}acetonitrile. Tan solid (3.80 g, 95%): ¹H NMR (CDCl₃, 400 MHz) δ8.04-8.05 (m, 1H), 7.67-7.70 (m, 1H), 7.72 (d, J=8.0 Hz, 1H), 6.99 (d, J=8.4 Hz, 1H), 6.94-6.98 (m, 2H), 3.78 (s, 5H), 3.69 (s, 2H). LCMS m/z (relative intensity) 280.0 [M+1]⁺ (100); 301.9 [M+Na]⁺ (10).

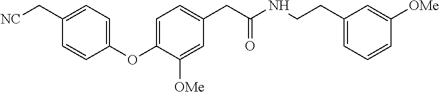 [SMILES: COc1cccc(CCNC(=O)Cc2ccc(Oc3ccc(CC#N)cc3)c(OC)c2)c1]

Cyano. Amber oil (1.67 g, ¹H NMR (CDCl₃, 400 MHz) δ7.23-7.26 (m, 2H), 7.17 (t, J=8.0 Hz, 1H), 6.90-6.93 (m, 3H), 6.83 (d, J=2.0 Hz, 1H), 6.72-6.76 (m, 2H), 6.64-6.66 (m, 2H), 5.47 (br t, 1H), 3.78 (s, 3H), 3.77 (s, 3H), 3.71 (s, 2H), 3.48-3.53 (m, 4H), 2.75 (t, J=6.8 Hz, 2H). LCMS m/z (relative intensity) 431.0 [M+1]⁺ (100).

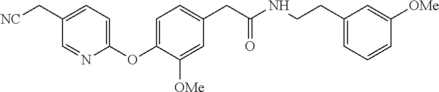 [SMILES: COc1cccc(CCNC(=O)Cc2ccc(Oc3ccc(CC#N)cn3)c(OC)c2)c1]

Cyano. Amber oil (1.21 g, 92%): ¹H NMR (CDCl₃, 400 MHz) δ8.04-8.05 (m, 1H), 7.66-7.69 (m, 1H), 7.17 (t, J=8.0 Hz, 1H), 7.06 (d, J=8.0 Hz, 1H), 6.96 (d, J=8.4 Hz, 1H), 6.84 (d, J=2.0 Hz, 1H), 6.79 (dd, J=8.0, 2.0 Hz, 1H), 6.73-6.76 (m, 1H), 6.64-6.67 (m, 2H), 5.56 (br t, 1H), 3.77 (s, 3H), 3.71 (s, 3H), 3.69 (s, 2H), 3.53 (s, 2H), 3.49 (q, J=6.8 Hz, 2H), 2.75 (t, J=6.8 Hz, 2H). ¹³C NMR (CDCl₃, 100 MHz) δ170.66, 163.48, 159.82, 151.88, 146.60, 141.39, 140.28, 138.97, 132.96, 129.62, 123.34, 121.92, 121.03, 120.11 117.19, 114.46, 113.85, 111.76, 111.20, 55.88, 55.14, 43.77, 40.65, 35.46, 20.43 ppm. LCMS m/z (relative intensity) 432.1 [M+1]⁺ (100).

General procedure for the preparation of di-acids.

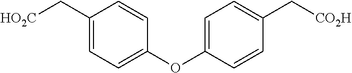 [SMILES: O=C(O)Cc1ccc(Oc2ccc(CC(=O)O)cc2)cc1]

Preparation of 2,2′-(oxydibenzene-4 1-diyl)diacetic acid. To 2,2′-(oxydibenzene-4,1-diyl)diacetonitrile (0.52 g, 2.09 mmol) was added 30% HCl (10 mL). The resulting suspension was stirred (100° C., 221w). The reaction mixture was cooled (RT) and diluted with H₂O (10 mL). The solid is collected via filtration, washed with H₂O (10 mL) and dried under high vacuum to provide the title compound as a tan solid (0.51 g, 84%): ¹H NMR (DMSO-d₆, 400 MHz) δ12.28 (br s, 2H), 7.20-7.23 (m, 4H) 6.89-6.92 (m, 4H), 3.50 (s, 4H) ¹³C NMR (DMSO-d₆, 100 MHz) δ173.18, 155.87, 131.38, 130.47, 118.77, 40.25 ppm. LCMS m/z (relative intensity) 285.0 [M−1]⁻ (100).

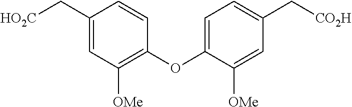 [SMILES: COc1cc(CC(=O)O)ccc1Oc1ccc(CC(=O)O)cc1OC]

2,2′-[Oxybis(3-methoxy-4,1-phenylene)]diacetic acid. White foam (4.16 g, 99%): ¹H NMR (DMSO-d₆, 400 MHz) 12.31 (br s, 1H), 7.01 (d, J=1.6 Hz, 1H), 6.75 (dd, J=8.0, 2.0 Hz, 1H), 6.63 (d, J=8.0 Hz, 1H), 3.76 (s, 3H), 3.54 (s, ²H). LCMS m/z (relative intensity) 347.0 [M+1]⁺ (30).

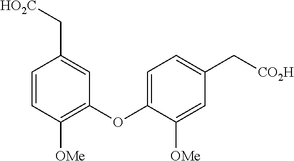 [SMILES: COc1cc(CC(=O)O)ccc1Oc1cc(CC(=O)O)ccc1OC]

{4-[5-(Carboxymethyl)-2-methoxyphenoxy]-3-methoxyphenyl}acetic acid. Tan foam (18.42 g, 100%): NMR (DMSO-d₆, 400 MHz) δ12.27 (br s, 2H), 7.04 (d, J=8.0 Hz, 1H), 7.02 (d, J=2.0 Hz, 1H), 6.95 (dd, J=8.0, 2.0 Hz, 1H), 6.76 (dd, J=8.0, 2.0 Hz, 1H), 6.64-6.66 (m, 2H), 3.76 (s, 6H), 3.54 (s, 2H), 3.43 (s, 2H). ¹³C NMR (DMSO-d₆, 100 MHz) δ 173.13, 150.08, 149.29, 149.28, 144.34, 131.10, 128.04, 125.07, 122.11, 119.66, 118.32, 114.82, 113.27, 56.11, 56.05, 40.69, 40.53 ppm. LCMS m/z (relative intensity) 301.0 [M−CO₂H]⁺ (100), 347.0 [M+1]⁺ (70); 369.0 [M+Na]⁺ (35).

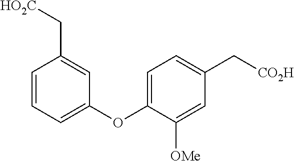 [SMILES: COc1cc(CC(=O)O)ccc1Oc1cccc(CC(=O)O)c1]

{3-[4-(Carboxymethyl)-2-methoxyphenoxy]phenyl}acetic acid. White solid (2.85 g, 81%): NMR (DMSO-d₆, 400 MHz) δ12.30 (hr s, 2H), 7.22 (t, J=8.0 Hz, 1H), 7.08 (d, J=1.7 Hz, 1H), 6.97 (d, J=8.0 Hz, 1H), 6.90 (d, J=8.0 Hz, 1H), 6.86 (dd, J=8.0, 1.7 Hz, 1H), 6.77-6.78 (m, 1H), 6.66 (dd, J=8.0, 2.4 Hz, 1H), 3.72 (s, 3H), 3.59 (s, 2H), 3.52 (s, 2H). ¹³C NMR (DMSO-d₆, 100 MHz) δ173.03, 172.87, 158.23, 151.42, 142.36, 137.13, 132.97, 129.77, 123.52, 122.41, 121.87, 117.39, 115.03, 114.34, 56.01, 40.94, 40.80 ppm. LCMS m/z (relative intensity) 317.0 [M+1]⁺ (50), 271.0 [M−CO₂H]⁺ (90); 338.9 [M+Na]⁺ (100).

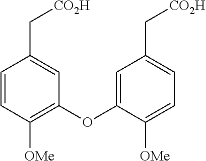 [SMILES: COc1ccc(CC(=O)O)cc1Oc1cc(CC(=O)O)ccc1OC]

2,2′-[Oxybis(4-methoxybenzene-3,1-diyl)]diacetic acid. Tan semisolid (1.11 g, 82%): ¹H NMR (CDCl₃, 400 MHz) δ7.96 (dd, J=8.1, 1.8 Hz, 1H), 6.91 (d, J=8.1 Hz, 1H), 6.78 (d, J=1.8 Hz, 1H), 3.84 (s, 3H), 3.49 (s, 2H). LCMS m/z (relative intensity) 347.0 [M+1]⁺ (90); 368.9 [M+Na]⁺ (100).

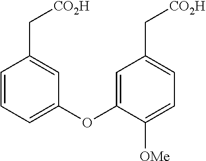 [SMILES: COc1ccc(CC(=O)O)cc1Oc1cccc(CC(=O)O)c1]

{3-[5-(Carboxymethyl)-2-methoxyphenoxy]phenyl}acetic acid. Amber oil (0.82 g, 84%): ¹H NMR (DMSO-d₆, 400 MHz) δ12.23 s, 2H), 7.16 (t, J=8.0 Hz, 1H), 7.01-7.06 (m, 2H), 6.89 (d, J=1.2 Hz, 1H), 6.85 (d, J=8.0 Hz, 1H), 6.74 (br t, J=2.0 Hz, 1H), 6.58-6.61 (m, 1H), 3.66 (s, 3H), 3.47 (s, 2H), 3.45 (s, 2H). ¹³C NMR (DMSO-d₆, 100 MHz) δ173.11, 172.85, 158.12, 150.56, 143.26, 137.11, 129.76, 128.35, 126.82, 123.56, 123.21, 117.53, 114.29, 113.61, 56.10, 40.91, 39.94 ppm. LCMS m/z (relative intensity) 317.0 [M+1]⁺ (35), 271.0 [M−C(O)OH]⁺ (100); 339.0 [M+Na]⁺ (35).

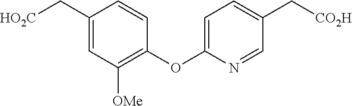 [SMILES: COc1cc(CC(=O)O)ccc1Oc1ccc(CC(=O)O)cn1]

{6-[4-(Carboxymethyl)-2-methoxyphenoxy]pyridine-3-yl}acetic acid. Tan solid (3.63 g, 90%): ¹H NMR (DMSO-d₆, 400 MHz) δ7.87 (d, J=2.4 Hz, 1 7.64 (dd, J=8.4, 2.4 Hz, 1H), 6.98-7.00 (m, 2H), 6.86 (d, J=8.4 Hz, 1H), 6.81 (dd, J=8.2, 1.8 Hz, 1H), 3.63 (s, 3H), 3.55 (s, 2H), 3.51 (s, 2H). ¹³C NMR (DMSO-d₆, 100 MHz) δ 173.08, 172.95, 162.47, 151.63, 147.64, 141.37, 141.01, 133.28, 125.57, 123.18, 122.08, 114.65, 110.05, 55.96, 40.85, 37.05 ppm. LCMS m/z (relative intensity) 318.0 [M+1]⁺ (100).

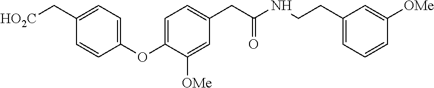 [SMILES: COc1cccc(CCNC(=O)Cc2ccc(Oc3ccc(CC(=O)O)cc3)c(OC)c2)c1]

Acid. Amber oil (1.67 g, 82%): ¹H NMR (CDCl₃, 400 MHz) δ7.20-7.22 (m, 2H), 7.16 (t, J=8.0 Hz, 1H), 6.86-6.89 (m 3H), 6.80 (d, J=2.0 Hz, 1H), 6.72-6.75 (m, 1H), 6.68 (dd, J=8.4, 2.0 Hz, 1H), 6.62-6.64 (m, 2H), 5.50 (br t, 1H), 3.75-3.76 (m, 7H), 3.62 (t, J=6.0 Hz, 1H), 3.47-3.54 (m, 4H), 2.73 (t, J=6.8 Hz, 2H). LCMS m/z (relative intensity) 450.0 [M+1]⁺ (100).

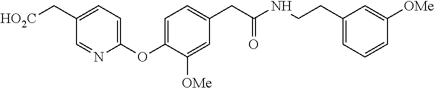 [SMILES: COc1cccc(CCNC(=O)Cc2ccc(Oc3ccc(CC(=O)O)cn3)c(OC)c2)c1]

Acid. Amber oil (1.02 g, 81%): ¹H NMR (DMSO-d₆, 400 MHz) δ12.37 (hr s, 1H), 8.26 (br s, 1H), 8.08 (br t, 1H), 7.85 (br s, 1H), 7.62 (d, J=8.4 Hz, 1H), 7.13 (t, J=8.0 Hz, 1H), 6.92-6.98 (m, 2H), 6.69-6.86 (m, 4H), 3.66 (s, 3H), 3.59 (s. 3H), 3.49 (s, 2H), 3.34 (s, 2H), 3.23-3.28 (m, 2H), 2.64 (t, J=7.0 Hz, 2H). ¹³C NMR (DMSO-d₆, 100 MHz) δ173.06, 172.94, 170.33, 162.47, 159.66, 151.57, 147.64, 141.35, 141.01, 134.70, 129.69, 125.52, 123.11, 122.08, 121.31, 114.62, 114.21, 111.96, 110.05, 55.89, 55.27, 42.61, 40.83, 37.00, 35.49 ppm. LCMS m/z (relative intensity) 451.1 [M+1]⁺ (100).

General procedure for the preparation of bis-amides.

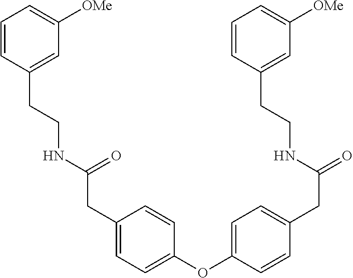 [SMILES: COc1cccc(CCNC(=O)Cc2ccc(Oc3ccc(CC(=O)NCCc4cccc(OC)c4)cc3)cc2)c1]

Preparation of bis-amide. To a suspension of 2,2′-(oxydibenzene-4,1-diyl)diacetic acid (0.48 g, 1.68 mmol), 2-(3-methoxyphenyl)ethanamine (0.51 g, 3.37 mmol), HOBt (0.68 g, 5.05 mmol) and TEA (0.51 g, 5.05 mmol) in DMF (4 mL) was added EDCl (0.97 g, 5.05 mmol). The resulting suspension was stirred (70° C., 19 hr). The reaction mixture was cooled (RT) and partitioned with EtOAc (20 mL) and H₂O (20 mL). The aqueous layer was extracted with ECO Ac (20 mL). The combined organic layers were washed with 1 M HCl (2×20 mL), saturated NaHCO₃ (2×20 mL), dried (Na₂SO₄), filtered and concentrated in vacuo to provide the title compound as a tan solid (0.78 g, 83%): LCMS m/z (relative intensity) 553.0 [M+1]⁺ (100).

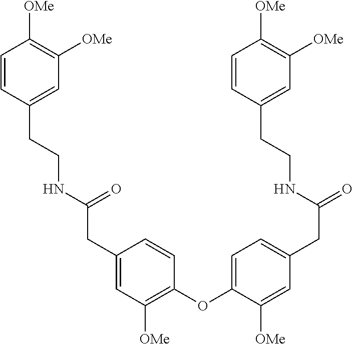 [SMILES: COc1ccc(CCNC(=O)Cc2ccc(Oc3ccc(CC(=O)NCCc4ccc(OC)c(OC)c4)cc3OC)c(OC)c2)cc1OC]

Bis-Amide. Tan foam (7.8 g, 100%): ¹H NMR (CDCl₃, 400 MHz) δ6.79 (d, J=1.6 Hz, 1H), 6.75 (dd, J=8.4, 0.8 Hz, 2H), 6.66 (dd, J=9.4, 1.8 Hz, 2H), 6.53 (dd, J=8.0, 2.0 Hz, 1H), 5.48 (br t, 1H), 3.85 (s, 3H), 3.84 (s, 3H), 3.80 (s, 3H), 3.44-3.50 (m, 4H), 2.70 (t, J=6.8 Hz, 2H). LCMS m/z (relative intensity) 673.3 [M+1]⁺ (100).

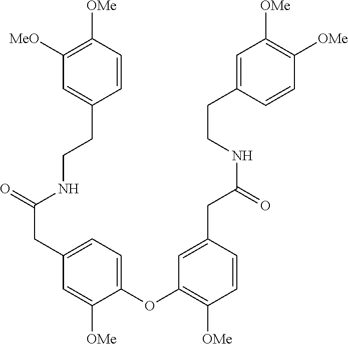 [SMILES: COc1ccc(CCNC(=O)Cc2ccc(Oc3cc(CC(=O)NCCc4ccc(OC)c(OC)c4)ccc3OC)c(OC)c2)cc1OC]

Bis-amide. Tan foam (4.02 g, 100%): ¹H NMR (CDCl₃, 400 MHz) 86.87-6.91 (m, 2H), 6.72-6.79 (m, 2H), 6.62-6.71 (m, 6H), 6.52-6.57 (m, 2H), 5.44-5.50 (m, 2H), 3.83-3.86 (m, 15H), 3.79 (s, 3H), 3.39-3.49 (m, 8H), 2.64-2.72 (m, 4H). ¹³C NMR (CDCl₃, 100 MHz) δ170.87, 170.83, 150.58, 149.85, 149.05, 147.67, 145.81, 144.97, 131.11, 131.04, 130.64, 127.47, 124.90, 121.71, 120.66, 120.64, 119.95, 118.74, 113.57, 112.86, 111.75, 111.67, 111.26, 111.20, 56.01, 55.91, 55.88, 55.83, 43.60, 42.98, 40.80, 35.16, 35.02 ppm, the remaining peaks were not detected and are expected to overlap with the peaks at 111.75, 55.88 and 40.80 ppm. LCMS m/z (relative intensity) 673.3.0 [M+1]⁺ (100).

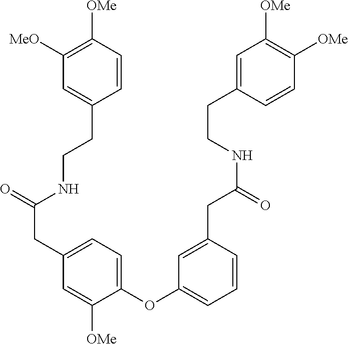 [SMILES: COc1ccc(CCNC(=O)Cc2cccc(Oc3ccc(CC(=O)NCCc4ccc(OC)c(OC)c4)cc3OC)c2)cc1OC]

Bis-Amide. Tan foam (3.61 g, 98%): ¹H NMR (CDCl₃, 400 MHz) δ7.24 (t, J=8.0 Hz, 1H), 6.88 (dd, J=8.0, 1.2 Hz, 3H), 6.80-6.84 (m, 2H), 6.71-6.76 (m, 3H), 6.64-6.66 (m, 2H), 6.56-6.59 (m, 2H), 5.59 (hr s, 1H), 5.57 (br s, 1H), 3.83-3.86 (m, 12H), 3.77-3.78 (m, 3H), 3.41-3.51 (m, 8H), 2.72 (t, J=7.0 Hz, 2H), 2.68 (t, J=6.8 Hz, 2H). ¹³C NMR (CDCl₃, 100 MHz) δ 170.69, 170.51, 158.21, 151.55, 149.06, 149.04, 147.68, 147.65, 143.80, 136.59, 131.86, 131.17, 131.04, 130.09, 123.55, 121.90, 121.32, 120.64, 118.80, 115.86, 113.86, 111,75, 111.72, 111.34, 111.20, 56.92, 55.91, 55.83, 43.71, 43.57, 40.90, 40.78, 35.15, 35.02 ppm, the remaining peaks were not detected and are expected to overlap with the peaks at 111.75 and 55.90 ppm. LCMS m/z (relative intensity) 643.2 [M+1]⁺ (100); 665.2 [M+Na]⁺ (20).

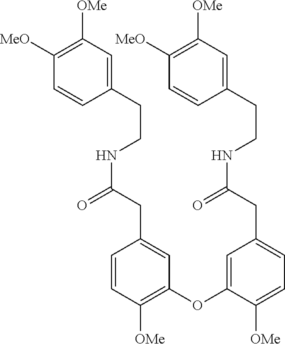 [SMILES: COc1ccc(CCNC(=O)Cc2ccc(OC)c(Oc3cc(CC(=O)NCCc4ccc(OC)c(OC)c4)ccc3OC)c2)cc1OC]

Bis-amide. Tan solid (1.70 g, 100%): ¹H NMR (CDCl₃, 400 MHz) δ6.86-6.92 (m, 2H), 6.74 (d, J=8.1 Hz, 1H), 6.62-6.63 (m, 2H), 6.54 (dd, J=8.1, 2.0 Hz, 1H), 5.47 (br t, 1H), 3.85 (s, 6H), 3.83 (s, 3H), 3.37-3.43 (m, 4H), 2.65 (t, J=7.0 Hz, 2H). LCMS m/z (relative intensity) 673.2 [M+1]⁺ (100); 695.3 [M+Na]⁺ (10).

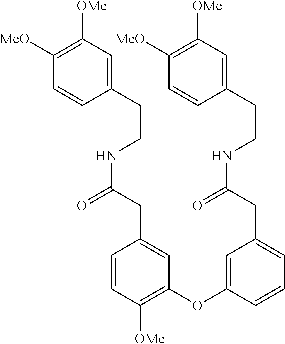 [SMILES: COc1ccc(CCNC(=O)Cc2cccc(Oc3cc(CC(=O)NCCc4ccc(OC)c(OC)c4)ccc3OC)c2)cc1OC]

Bis-amide. Tan solid (1.70 g, 98%): ¹H NMR (CDCl₃, 400 MHz) δ7.22 (t, J=8.0 Hz, 1H), 6.92-6.98 (m, 3H), 6.87 (d, J=7.6 Hz, 2H), 6.76-6.81 (m, 2H), 6.72-6.75 (m, 2H), 6.63-6.64 (m, 2H), 6.56 (dd, J=8.0, 1.8 Hz, 1H), 5.55-5.60 (m, 2H), 3.84 (s, 3H), 3.82 (s, 6H), 3.81 (s, 6H), 3.40-3.46 (m, 8H), 3.66-3.70 (m, 4H). ¹³C NMR (CDCl₃, 100 MHz) δ170.82, 170.52, 158.13, 150.58, 149.04, 149.01, 147.67, 147.62, 144.69, 136.59, 131.20, 131.12, 130.12, 127.86, 125.83, 123.59, 122.09, 120.68, 120.64, 118.11, 115.87, 113.14, 111.77, 111.76, 111.35, 111.24, 56.02, 55.87, 55.83, 55.82, 43.73, 42.86, 40.92, 40.79, 35.14, 35.08, 35.01 ppm; the remaining peak was not detected and believed to be overlap with the peaks at 55.82 ppm. LCMS m/z (relative intensity) 643.2 [M+1]⁺ (100).

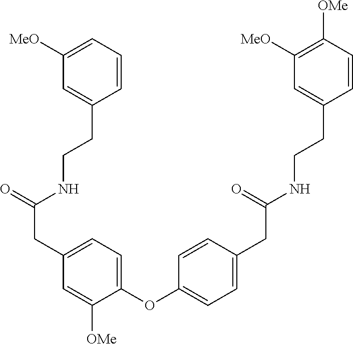 [SMILES: COc1cccc(CCNC(=O)Cc2ccc(Oc3ccc(CC(=O)NCCc4ccc(OC)c(OC)c4)cc3)c(OC)c2)c1]

Bis-amide. Tan solid (0.87 g, 92%): ¹H NMR (CDCl₃, 400 MHz) δ7.17 (t, J=8.0 Hz, 1H), 7.10 (d, J=8.4 Hz, 2H), 6.86-6.91 (m, 3H), 6.83 (d, J=2.0 Hz, 1H), 6.71-6.77 (m, 3H), 6.64-6.66 (m, 3H), 6.56 (dd, J=8.4, 1.8 Hz, 1H), 5.46 (br t, 1H), 5.41 (hr t, 1H), 3.81-3.85 (m, 6H), 3.75-3.79 (m, 6H), 3.43-3.53 (m, 8H), 2.75 (t, J=6.6 Hz, 2H), 2.69 (t, J=6.8 Hz, 2H). LCMS m/z (relative intensity) 613.2 [M+1]⁺ (100); 635.1 [M+Na]⁺ (10).

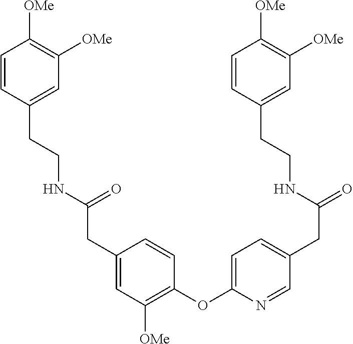 [SMILES: COc1ccc(CCNC(=O)Cc2ccc(Oc3ccc(CC(=O)NCCc4ccc(OC)c(OC)c4)cc3OC)nc2)cc1OC]

Bis-amide. Tan solid (1.15 g, 61%): ¹H NMR (CDCl₃, 400 MHz) δ7.92 (d, J=2.4 Hz, 1H), 7.56 (dd, J=8.4, 2.4 Hz, 1H), 7.06 (d, J=8.0 Hz, 1H), 6.89 (d, J=8.4 Hz, 1H), 6.82 (d, J=1.6 Hz, 1H), 6.74-6.80 (m, 3H), 6.66 (d, J=1.6 Hz, 2H), 6.60 (dd, J=8.0, 2.0 Hz, 1H), 6.56 (dd, J=8.0, 2.0 Hz, 1H), 5.55 (br t, 1H), 5.50 (br t, 1H), 3.86 (s, 3H), 3.84 (s, 9H), 3.71 (s, 3H), 3.43-3.53 (m, 8H), 2.72 (t, J=6.8 Hz, 4H). ¹³C NMR (CDCl₃, 100 MHz) δ170.79, 170.14, 162.88, 151.99, 149.11, 149.04, 147.75, 147.65, 147.43, 141.65, 140.37, 132.77, 131.13, 131.06, 124.98, 123.40, 121.92, 120.68, 120.64, 113.91, 111.78, 111.71, 111.34, 111.28, 110.87, 55.94, 55.88, 55.86, 55.85, 43.74, 40.92, 40.86, 39.77, 35.09, 35.01 ppm; the remaining peak was not detected and believed to be overlap with the peaks at 55.88 ppm. LCMS m/z (relative intensity) 644.1 [M+1]⁺ (100).

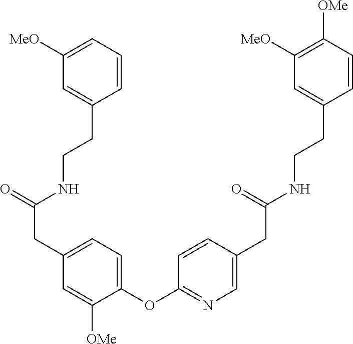 [SMILES: COc1cccc(CCNC(=O)Cc2ccc(Oc3ccc(CC(=O)NCCc4ccc(OC)c(OC)c4)cn3)c(OC)c2)c1]

Bis-Amide. Tan foam (1.00 g, 73%): ¹H NMR (CDCl₃, 400 MHz) δ7.91 (d, J=2.0 Hz, 7.54-7.58 (m, 1H), 7.17 (t, J=8.2 Hz, 1H), 7.05 (dd, J=7.8, 2.6 Hz, 1H), 6.87-6.89 (m, 6.82-6.84 (m, 1H), 6.73-6.79 (m, 4H), 6.64-6.67 (m, 2H), 6.54-6.62 (m, 1H), 5.61 (br t, 1H), 5.59 (br t, 1H), 3.85 (s, 3H), 3.84 (s, 3H), 3.77 (s, 3H), 3.70-3.71 (m, 3H), 3.52 (s, 2H), 3.44-3.51 (1n, 4H), 3.42 (s, 2H), 2.70-2.76 (m, 4H). ¹³C NMR (CDCl₃, 100 MHz) δ170.73, 170.12, 162.91, 159.80, 151.97, 149.11, 147.66, 147.49, 141.65, 140.33, 132.76, 131.09, 129.62, 124.91, 123.37, 121.91, 121.04, 120.67, 114.45, 113.89, 111.74, 111.69, 111.34, 111.27, 110.85, 55.92, 55.89, 55.86, 55.84, 43.75, 40.90, 40.67, 39.79, 35.47, 35.09 ppm. LCMS m/z (relative intensity) 644.2 [M+1]⁺ (100).

General procedure for the preparation of bis-DHIQs.

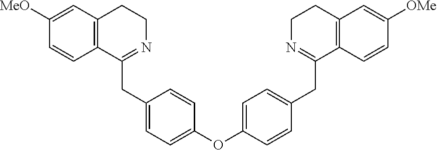 [SMILES: COc1ccc2c(c1)CCN=C2Cc1ccc(Oc2ccc(CC3=NCCc4cc(OC)ccc43)cc2)cc1]

Preparation of bis-DHIQ. To a suspension of bis-amide (0.76 g, 1.38 mmol) and ACN (3.0 mL) was added phosphorus oxychloride (2.11 g, 13.75 mmol, 1.30 mL). The resulting suspension was stirred (80° C., 1.2.5 hr). The reaction mixture was cooled (RT) and concentrated in vacuo. The resulting residue was partitioned with EtOAc (20 mL) and H₂O (20 mL). The biphasic reaction mixture was stirred vigorously and treated with solid NaHCO₃ (slow addition) until the aqueous phase is basic. The organic layer was separated and the aqueous layer was extracted with EtOAc (20 mL). The combined organic layers were washed with saturated NaHCO₃ (2×40 mL), dried (Na₂SO₄), filtered and concentrated in vacuo to provide the title compound as a tan semi-solid (0.69 g, 98%): LCMS m/z (relative intensity) 517.1 [M+1]⁺ (30), 259.1 (100) [M+1]⁺/2 (100).

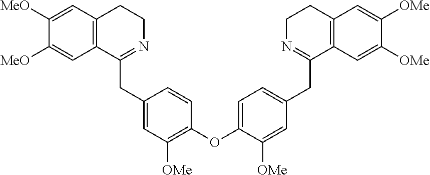 [SMILES: COc1cc2c(cc1OC)C(Cc1ccc(Oc3ccc(CC4=NCCc5cc(OC)c(OC)cc54)cc3OC)c(OC)c1)=NCC2]

Bis-DHIQ. Brown residue (3.23 g, 100%): NMR (CDCl₃, 400 MHz) δ6.96 (s, 1H), 6.90 (d, J=2.0 Hz, 1H), 6.77 (dd, J=8.4, 2.0 Hz, 1H), 6.66-6.69 (m, 2H), 3.99 (s, 2H), 3.89 (s, 3H), 3.72-3.76 (m, 8H), 2.65 (t, J=7.6 Hz, 2H). LCMS m/z (relative intensity) 637.3 [M+1]⁺ (35), 319.2 [M/2]⁺ (100).

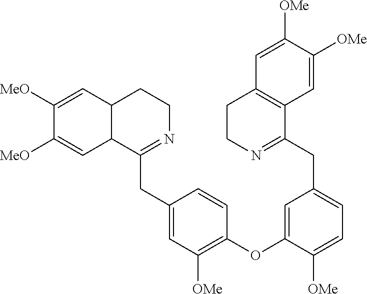 [SMILES: COC1=CC2CCN=C(Cc3ccc(Oc4cc(CC5=NCCc6cc(OC)c(OC)cc65)ccc4OC)c(OC)c3)C2C=C1OC]

Bis-DHIQ. Tan foam (8.1 g, 100%): ¹H NMR (CDCl₃, 400 MHz) δ6.96-6.98 (m, 2H), 6.85-6.90 (m, 3H), 6.73-6.77 (m, 2H), 6.63-6.67 (m, 3H), 4.01 (s, 2H), 3.89-3.92 (m, 9H), 3.72-3.80 (m, 11H), 3.61-3.65 (m, 4H), 2.65-2.69 (m, 2H), 2.54-2.58 (m, 2H). ¹³C NMR (CDCl₃, 100 MHz) δ165.56, 165.40, 150.81, 150.65, 150.47, 149.01, 147.29, 147.20, 145.91, 144.49, 133.80, 131.84, 131.80, 130.71, 123.54, 121.58, 121.43, 120.78, 119.02, 118.66, 112.69, 112.64, 110.28, 110.20, 109.67, 109.57, 56.04, 55.99, 55.94, 55.85, 47.16, 47.04, 43.24, 42.54, 25.83, 25.72 ppm; the remaining peak was not detected and believed to be overlap with the peaks at 55.94 ppm. LCMS m/z (relative intensity) 637.3 [M+1]⁺ (35), 319.1 [M/2]⁺ (100).

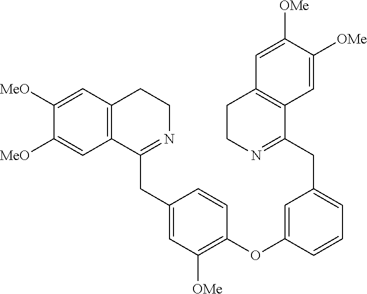 [SMILES: COc1cc2c(cc1OC)C(Cc1cccc(Oc3ccc(CC4=NCCc5cc(OC)c(OC)cc54)cc3OC)c1)=NCC2]

Bis-DHIQ. Amber oil (1.65 g, 96%).

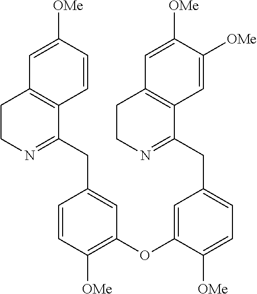 [SMILES: COc1ccc2c(c1)CCN=C2Cc1ccc(OC)c(Oc2cc(CC3=NCCc4cc(OC)c(OC)cc43)ccc2OC)c1]

Bis-DHIQ. Tan semisolid (0.90 g, 95%): LCMS m/z (relative intensity) 637.1 [M+1]⁺ (15), 319.2 [M+1]⁺/2 (100).

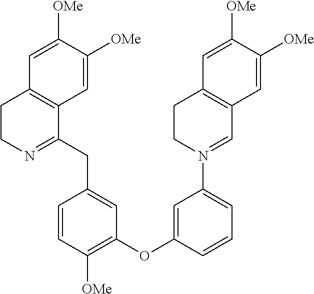

Bis-DHIQ. Tan foam (1.30 g, 100%).

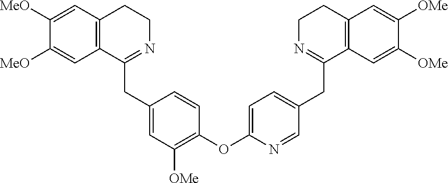 [SMILES: COc1cc2c(cc1OC)C(Cc1ccc(Oc3ccc(CC4=NCCc5cc(OC)c(OC)cc54)cc3OC)nc1)=NCC2]

Bis-DHIQ. Tan foam (1.56 g, 100%).

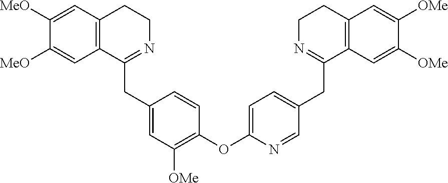 [SMILES: COc1cc2c(cc1OC)C(Cc1ccc(Oc3ccc(CC4=NCCc5cc(OC)c(OC)cc54)cc3OC)nc1)=NCC2]

Bis-DHIQ. Tan foam (0.85 g, 90%).

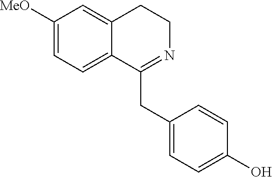 [SMILES: COc1ccc2c(c1)CCN=C2Cc1ccc(O)cc1]

4-[(6-Methoxy-3,4-dihydroisoquinolin-1-yl)methyl]phenol. Amber semisolid (1.32 g, 71%). LCMS m/z (relative intensity) 268.1 [M+1]⁺ (100).

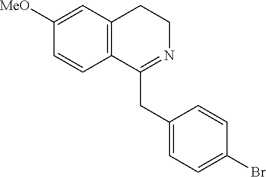 [SMILES: COc1ccc2c(c1)CCN=C2Cc1ccc(Br)cc1]

1-(4-Bromobenzyl)-6-methoxy-dihydroisoquinoline. Brown hygroscopic solid (2.32 g, 98%): ¹H NMR (CDCl₃, 400 MHz) δ7.81 (d, J=8.9, 1.5 Hz, 1H), 7.42 (dd, J=8.4, 2.8 Hz, 2H), 7.32 (d, J=8.4 Hz, 2H), 6.90 (dd, J=8.9, 2.4 Hz, 1H), 6.82 (d, J=1.5 Hz, 2H), 4.55 (s, 2H), 3.97-3.93 (m, 2H), 3.91 (s, 3H), 3.05-3.01 (m, 2H); LCMS m/z (relative intensity) 330.1 [M+1]⁺ (100), 332.1 (100).

General procedure for the preparation of bis-NH-THIQs.

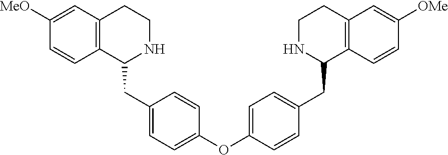

Preparation (R,R)-bis-NH-THIQ. To a solution of dichloro(p-cumene)ruthenium(II) dimer (16 mg, 0.027 mmol, 2 mol %), (1S,2S)-N-(4-tolylsulfonyl)-1,2-diphenylethylenediamine (20 mg, 0.053 mmol, 4 mol %) in DMF (2.3 mL) was added TEA (27 mg, 0.267 mmol, 20 mol %). The resulting solution was degassed by bubbling dry nitrogen into the solution. The solution was stirred (80° C., 1 hr). A solution of methoxy bis-DHIQ (0.69 mg, 1.34 mmol) in DMF (1.0 mL) is degassed by bubbling dry nitrogen into the solution. The resulting solution was treated with the catalyst solution and cooled (0° C.). The resulting solution was treated with HCO₂H:TEA azeotrope (5:2, 0.33 mL). The resulting solution was warmed (RT) and stirred (RT, 3 hr). The reaction mixture was partitioned with aqueous saturated NaHCO₃ (20 mL) and EtOAc (20 mL). The aqueous layer was extracted with EtOAc (2×20 mL). The combined organic layer were dried (Na₂SO₄), filtered and concentrated in vacuo to provide the title compound as a dark green residue (0.65 g, 99%): LCMS m/z (relative intensity) 521.1 [M+1]⁺ (20), 385.0 (100) [M+1]⁺/2 (100).

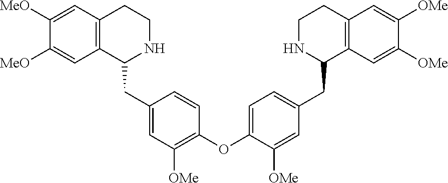

(R,R)-Bis-NH-THIQ. Green foam (2.27 g, 71%): LCMS m/z (relative intensity) 641.3 [M+1]⁺ (35), 321.2 [M+1]⁺/2 (100).

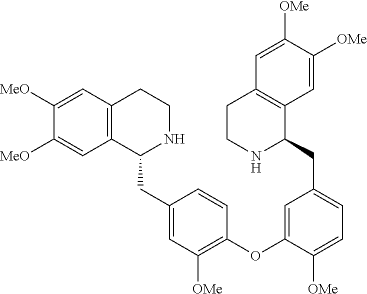 [SMILES: COc1cc2c(cc1OC)[C@@H](Cc1ccc(Oc3cc(C[C@H]4NCCc5cc(OC)c(OC)cc54)ccc3OC)c(OC)c1)NCC2]

(R,R)-Bis-NH-THIQ. Green foam (8.15 g, 100%): LCMS m/z (relative intensity) 641.2 [M+1]⁺ (35), 321.2 [M+1]⁺/2 (100).

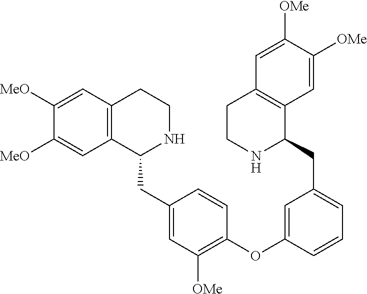 [SMILES: COc1cc2c(cc1OC)[C@@H](Cc1cccc(Oc3ccc(C[C@H]4NCCc5cc(OC)c(OC)cc54)cc3OC)c1)NCC2]

(R,R)-Bis-NH-THIQ. Amber oil (1.44 g, 87%).

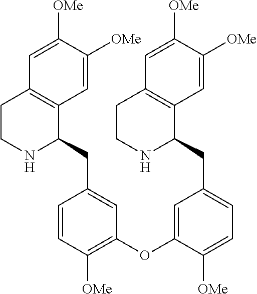 [SMILES: COc1cc2c(cc1OC)[C@@H](Cc1ccc(OC)c(Oc3cc(C[C@H]4NCCc5cc(OC)c(OC)cc54)ccc3OC)c1)NCC2]

(R,R)-Bis-NH-THIQ. Tan semisolid (0.76 g, 84%).

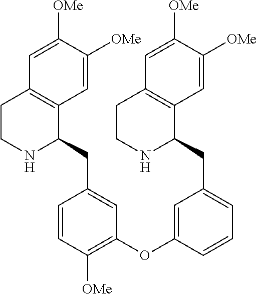 [SMILES: COc1cc2c(cc1OC)[C@@H](Cc1cccc(Oc3cc(C[C@H]4NCCc5cc(OC)c(OC)cc54)ccc3OC)c1)NCC2]

(R,R)-Bis-NH-THIQ. Amber oil (1.20 g, 92%).

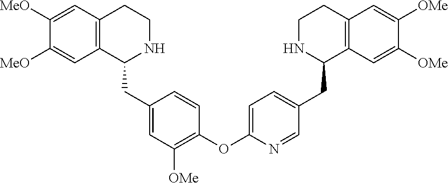 [SMILES: COc1cc2c(cc1OC)[C@@H](Cc1ccc(Oc3ccc(C[C@H]4NCCc5cc(OC)c(OC)cc54)cc3OC)nc1)NCC2]

(R,R)-Bis-NH-THIQ. Amber oil (0.77 g, 49%).

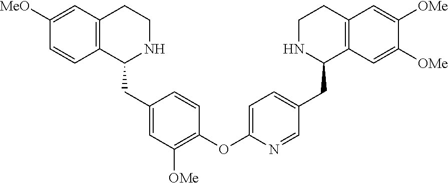 [SMILES: COc1ccc2c(c1)CCN[C@@H]2Cc1ccc(Oc2ccc(C[C@H]3NCCc4cc(OC)c(OC)cc43)cn2)c(OC)c1]

(R,R)-Bis-NH-THIQ. Amber oil (0.76 g, 89%).

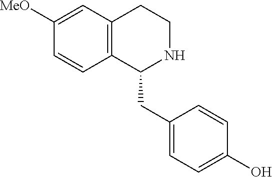 [SMILES: COc1ccc2c(c1)CCN[C@@H]2Cc1ccc(O)cc1]

4-{[(1R)-6-Methoxy-1,2,3,4-tetrahydroisoquinolin-1-yl]methyl}phenol. Tan semisolid (706 mg, 54%): LCMS m/z (relative intensity) 270.0 [M+1]⁺ (100).

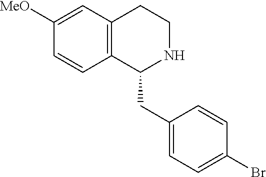 [SMILES: COc1ccc2c(c1)CCN[C@@H]2Cc1ccc(Br)cc1]

(1R)-1-(4-Bromobenzyl)-6-methoxy-1,2,3,4-tetrahydroisoquinoline. Dark green residue (1.45 g, 100%): LCMS m/z (relative intensity) 332.1 [M+1]⁺ (100), 334.1 (100).

General procedure for the preparation of (R,R)-bis-N-BOC-THIQs.

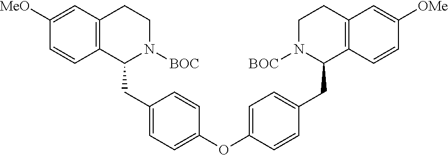 [SMILES: COc1ccc2c(c1)CCN(C(=O)OC(C)(C)C)[C@@H]2Cc1ccc(Oc2ccc(C[C@@H]3c4ccc(OC)cc4CCN3C(=O)OC(C)(C)C)cc2)cc1]

Preparation of (R,R)-bis-N-BOC-THIQ. To a solution of (R,R)-bis-THIQ (650 mg, 1.25 mmol) and TEA (505 mg, 5.0 mmol, 0.7 mL) in DCM (2.5 mL) was added BOC₂O (1.09 g, 5.00 mmol). The resulting solution was stirred (1 hr). The reaction was concentrated in vacuo and the resulting residue was partitioned with FAOAc (50 mL) and saturated NaHCO₃ (50 mL). The organic layer was washed with saturated NaHCO₃ (2×50 mL), dried (Na₂SO₄), filtered and concentrated in vacuo. The resulting residue was purified by flash column chromatography (SiO₂) using acetone:hexanes (0 to 50%) to provide the title compound as a white solid (277 mg, 31%): LCMS m/z (relative intensity) 743.0 [M+Na]⁺ (100), 741.4 [M−100]⁺ (100).

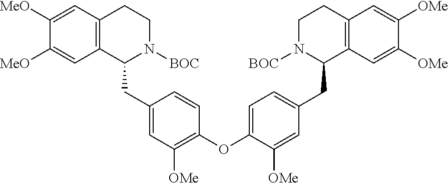 [SMILES: COc1cc2c(cc1OC)[C@@H](Cc1ccc(Oc3ccc(C[C@@H]4c5cc(OC)c(OC)cc5CCN4C(=O)OC(C)(C)C)cc3OC)c(OC)c1)N(C(=O)OC(C)(C)C)CC2]

(R,R)-Bis-N-BOC-THIQ. White Solid (1.72 g, 58%): ¹H NMR (CDCl₃, 400 MHz) shows a complex mixture of rotamers. LCMS m/z (relative intensity) 841.4 [M+1]⁺ (10), 741.4 [M−100]⁺ (100).

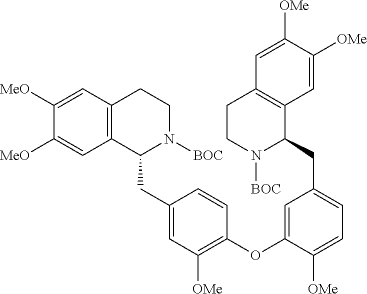 [SMILES: COc1cc2c(cc1OC)[C@@H](Cc1ccc(Oc3cc(C[C@@H]4c5cc(OC)c(OC)cc5CCN4C(=O)OC(C)(C)C)ccc3OC)c(OC)c1)N(C(=O)OC(C)(C)C)CC2]

(R,R)-Bis-N-BOC-THIQ. White Solid (7.64 g, 72%): ¹H NMR (CDCl₃, 400 MHz) shows a complex mixture of rotamers. LCMS m/z (relative intensity) 841.4 [M+1]⁺ (10).

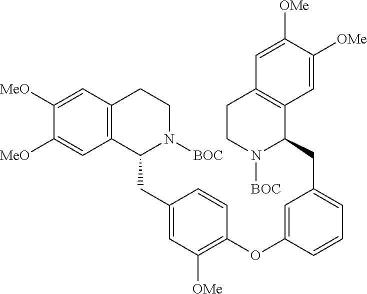 [SMILES: COc1cc2c(cc1OC)[C@@H](Cc1cccc(Oc3ccc(C[C@@H]4c5cc(OC)c(OC)cc5CCN4C(=O)OC(C)(C)C)cc3OC)c1)N(C(=O)OC(C)(C)C)CC2]

(R,R)-Bis-N-BOC-THIQ. White Solid (0.65 g, 34%): ¹H NMR (CDCl₃, 400 MHz) shows a complex mixture of rotamers. LCMS m/z (relative intensity) 711.2 [M-100]⁺ (100).

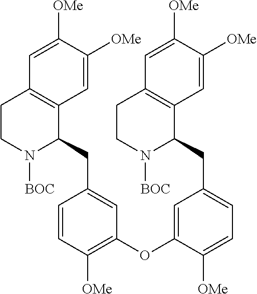 [SMILES: COc1cc2c(cc1OC)[C@@H](Cc1ccc(OC)c(Oc3cc(C[C@@H]4c5cc(OC)c(OC)cc5CCN4C(=O)OC(C)(C)C)ccc3OC)c1)N(C(=O)OC(C)(C)C)CC2]

(R,R)-Bis-N-BOC-THIQ. Tan solid (0.69 g, 69%): ¹ H NMR (CDCl₃, 400 MHz) shows a complex mixture of rotamers. LCMS m/z (relative intensity) 841.2 [M+1]⁺ (30); 741.2 [M−100]⁺ (100).

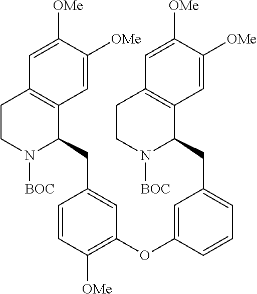 [SMILES: COc1cc2c(cc1OC)[C@@H](Cc1cccc(Oc3cc(C[C@@H]4c5cc(OC)c(OC)cc5CCN4C(=O)OC(C)(C)C)ccc3OC)c1)N(C(=O)OC(C)(C)C)CC2]

(R,R)-Bis-N-BOC-THIQ. White solid (0.94 g, 59%): ¹H NMR (CDCl₃, 400 MHz) shows a complex mixture of rotamers. LCMS m/z (relative intensity) 811.2 [M+1]⁺ (20), 711.2 [M+100]⁺ (100).

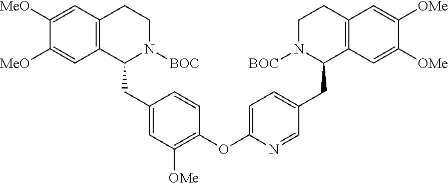 [SMILES: COc1cc2c(cc1OC)[C@@H](Cc1ccc(Oc3ccc(C[C@@H]4c5cc(OC)c(OC)cc5CCN4C(=O)OC(C)(C)C)cc3OC)nc1)N(C(=O)OC(C)(C)C)CC2]

(R,R)-Bis-N-BOC-THIQ. White solid (0.41 g, 20%): ¹H NMR (CDCl₃, 400 MHz) shows a complex mixture of rotamers. LCMS m/z (relative intensity) 812.2 [M+1]⁺ (100).

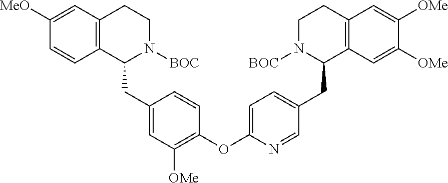 [SMILES: COc1ccc2c(c1)CCN(C(=O)OC(C)(C)C)[C@@H]2Cc1ccc(Oc2ccc(C[C@@H]3c4cc(OC)c(OC)cc4CCN3C(=O)OC(C)(C)C)cn2)c(OC)c1]

(R,R)-Bis-N-BOC-THIQ. White solid (0.36 g, 35%): ¹H NMR (CDCl₃, 400 MHz) shows a complex mixture of rotamers. LCMS m/z (relative intensity) 782.2 [M+1]⁺ (100).

General procedure for the preparation of (R)—N-formyl-THIQs.

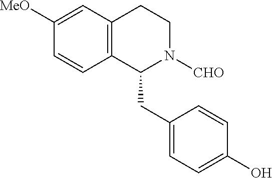 [SMILES: COc1ccc2c(c1)CCN(C=O)[C@@H]2Cc1ccc(O)cc1]

Preparation (1R)-1-[(4-hydroxyphenyl)methyl]-6-methoxy-3,4-dihydroisoquinoline-2(1H)-carbaldehyde. A solution of 4-{[(1R)-6-methoxy-1,2,3,4-tetrahydroisoquinolin-1-yl]methyl}phenol (680 mg, 2.53 mmol) in ethylformate (15 ml) was heated (65° C., 2 hr). The reaction mixture was concentrated in vacuo and the resulting residue was purified by flash column chromatography (SiO₂) using acetone:hexane (10-60%) to provide the title compound as a tan solid (403 mg, 54%): LCMS m/z (relative intensity) 298.0 [M+1]⁺ (100).

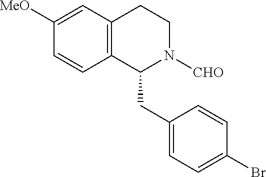 [SMILES: COc1ccc2c(c1)CCN(C=O)[C@@H]2Cc1ccc(Br)cc1]

(1R)-1-[(4-bromophenyl)methyl]-6-methoxy-3,4-dihydroisoquinoline-2(1H)-carbaldehyde. Amber oil (1.37 g, 100%): ¹H NMR (CDCl₃, 400 MHz) shows a complex mixture of rotamers. LCMS m/z (relative intensity) 359.9 [M+1]⁺ (100), 361.9 [M+2]⁺ (100).

General procedure for the preparation of (R,R)-bis-N-formyl-THIQs via Ullmann coupling.

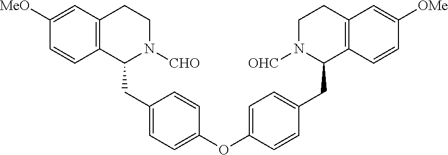 [SMILES: COc1ccc2c(c1)CCN(C=O)[C@@H]2Cc1ccc(Oc2ccc(C[C@@H]3c4ccc(OC)cc4CCN3C=O)cc2)cc1]

Preparation of (R,R)-bis-N-formyl-THIQ. To a solution of (1R)-1-[(4-bromophenyl)methyl]-6-methoxy-3,4-dihydroisoquinoline-2(1H)-carbaldehyde (50 mg, 0.14 mmol) and (1R)-1-[(4-hydroxyphenyl)methyl]-6-methoxy-3,4-dihydroisoquinoline-2(1H)-carbaldehyde (45 mg, 0.15 mmol) in pyridine (2 mL) in a microwave vial was added CuO (28 mg, 0.35 mmol) and K₂CO₃ (29 mg, 0.21 mmol). The resulting suspension was heated via microwave irradiation (210° C., 2 hr). The reaction was concentrated in vacuo and the resulting residue was partitioned with EtOAc (15 mL) and H₂O (15 mL). The organic layer was dried (Na₂SO₄), filtered and concentrated in vacuo. The resulting residue was purified by flash column chromatography (SiO₂) using acetone-hexanes (10-100%) to provide the target compound as a clear oil (33 mg, 41%).

General procedure for the preparation of (R,R)-bis-N-methyl-TIQs via reductive/animation.

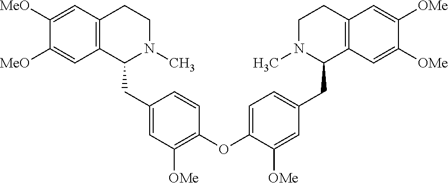 [SMILES: COc1cc2c(cc1OC)[C@@H](Cc1ccc(Oc3ccc(C[C@@H]4c5cc(OC)c(OC)cc5CCN4C)cc3OC)c(OC)c1)N(C)CC2]

Preparation of (R,R)-bis-N-methyl-THIQ. To a solution (R,R)-bis-N-BOC-THIQ (1.0 g, 1.19 mmol) in MeOH (5 mL) and DCM (5 mL) was added 4 M HCl-dioxane (4.0 ml). The resulting solution was stirred (2 hr). The reaction mixture was treated with 4 M HCl-dioxane (2.0 ml) and stirred (1 hr). The reaction was concentrated in vacuo and the resulting residue was partitioned with EtOAc (50 mL) and saturated NaHCO₃ (50 mL). The organic layer was washed with saturated NaHCO₃ (2×50 mL), dried (Na₂SO₄), filtered and concentrated in vacuo to provide the diamine intermediate as a white foam (650 mg, 85%). To a solution of the diamine (650 mg, 1.01 mmol) in DCL (4 mL) was added formaldehyde (37% in H₂O, 0.18 mL) and the resulting biphasic suspension was stirred (0.75 hr). The reaction mixture was treated with NaBH(OAc)₃ (473 mg, 2.23 mmol) and the resulting suspension was stirred (1.5 hr). The reaction mixture was concentrated in vacuo and the resulting residue was partitioned with EtOAc (50 mL) and saturated NaHCO₃ (50 mL). The organic layer was washed with saturated NaHCO₃ (2×50 mL), dried (Na₂SO₄), filtered and concentrated in vacuo. The resulting residue was purified by flash column chromatography (SiO₂) using TEA-ACN (1%) to provide the target compound as a white solid (715 mg, 74%; corrected yield from additional 250 mg reaction): ¹H NMR (CDCl₃, 400 MHz) δ 6.68 (br s, 1H), 6.66 (m, 1H), 6.58-6.61 (m, 1H), 6.56 (br s, 1H), 6.11 (s, 1H), 3.84 (s, 3H), 3.76 (s, 3H), 3.70-3.73 (m, 1H), 3.62 (s, 3H), 3.13-3.20 (m, 2H), 2.73-2.85 (m, 3H), 2.57-2.60 (m, 1H), 2.55 (s, 3H). ¹³C NMR (CDCl₃, 100 MHz) δ150.26, 147.56, 146.65, 144.58, 135.93, 129.53, 126.40, 122.19, 118.54, 114.35, 111.56, 111.37, 65.97, 56.34, 55.92, 47.45, 43.19, 42.89, 41.45, 25.87 ppm; additional peaks are believed to be rotamers. LCMS m/z (relative intensity) 669.3 [M+1]⁺ (30), 335.2 [M/2]⁺ (100).

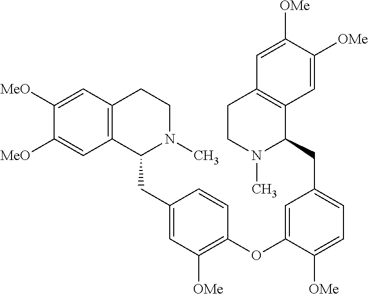 [SMILES: COc1cc2c(cc1OC)[C@@H](Cc1ccc(Oc3cc(C[C@@H]4c5cc(OC)c(OC)cc5CCN4C)ccc3OC)c(OC)c1)N(C)CC2]

(R,R)-Bis-N-methyl-THIQ. White solid (4.12 g, 81%): ¹H NMR (CDCl₃, 400 MHz) 86.84 (d, J=8.4 Hz, 1H), 6.74 (dd, J=8.4, 2.0 Hz, 1H), 6.66 (br s, 2H), 6.59-6.62 (m, 2H), 6.56 (s, 1H), 6.52 (s, 1H), 6.11 (s, 1H), 6.03 (s, 1H), 3.83 (s, 3H), 3.82 (s, 3H), 3.81 (s, 3H), 3.76 (s, 3H), 3.70-3.73 (m, 1H), 3.60-3.63 (m, 1H), 3.61 (s, 3H), 3.59 (s, 3H), 3.01-3.18 (m, 4H), 2.68-2.83 (m, 6H), 2.55 (s, 3H), 2.53-2.59 (m, 2H), 2.47 (s, 3H). LCMS m/z (relative intensity) 669.3 [M+1]⁺ (30), 335.2 [M/2]⁺ (100).

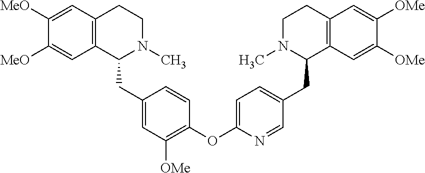 [SMILES: COc1cc2c(cc1OC)[C@@H](Cc1ccc(Oc3ccc(C[C@@H]4c5cc(OC)c(OC)cc5CCN4C)cc3OC)nc1)N(C)CC2]

(R,R)-Bis-N-methyl-THIQ. White solid (2.53 g, 65%): NMR (CDCl₃, 400 MHz) δ7.82 (br s, 1H), 7.32 (d, J=8.0 Hz, 1H), 6.98 (d, J=8.0 Hz, 1H), 6.73-6.76 (br t, 2H), 6.70 (br s, 1H), 6.57 (br s, 1H), 6.52 (br s, 1H), 6.25 (br s, 1H), 6.13 (br s, 1H), 3.84 (s, 3H), 3.83 (s, 3H), 3.71-3.75 (m, 4H), 3.64-3.66 (m, 7H), 3.09-3.21 (m, 3H), 2.98-3.03 (m, 1H), 2.70-2.88 (m, 6H), 2.59-2.63 (m, 1H), 2.55 (s, 3H), 2.49 (br s, 4H). ¹³C NMR (CDCl₃, 100 MHz) shows a complex mixture of rotamers. LCMS m/z (relative intensity) 640.3 [M+1]⁺ (35), 320.7 [M/2]⁺ (100).

General procedure for the preparation of bis-N-methyl-THIQs via LAH reduction.

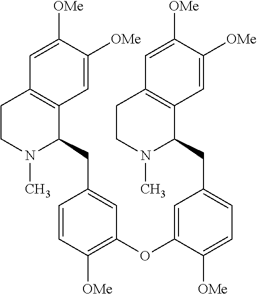 [SMILES: COc1cc2c(cc1OC)[C@@H](Cc1ccc(OC)c(Oc3cc(C[C@@H]4c5cc(OC)c(OC)cc5CCN4C)ccc3OC)c1)N(C)CC2]

Preparation of (R,R)-bis-N-methyl-THIQ. To a solution of (R,R)-bis-N-BOC-THQ (2.80 g, 3.33 mmol) in THF (30 mL) was added 1 M LAH-THF (9.99 ml, 9.99 mmol) in a dropwise manner. The resulting solution was stirred (0.5 hr) followed by heating (60° C., 20 hr). The reaction mixture was treated with water (7.0 mL), 15% aqueous NaOH (12 mL) and EtOAc (70 mL). The reaction mixture was filtered (Celite) and the residue was washed with additional EtOAc (15 mL). The filtrate was dried (Na₂SO₄), filtered and concentrated in vacuo. The resulting residue was purified by flash column chromatography (SiO₂) using TEA-ACN (1%) to provide the target compound as a white solid (1.39 g, 62%): ¹H NMR (CDCl₃, 400 MHz) δ6.82 (d, J=8.4 Hz, 1H), 6.70 (d, J=8.4, 2.0 Hz, 1H), 6.66 (d, J=2.0 Hz, 1H), 6.51 (s, 1H), 6.05 (s, 1H), 3.79-3.84 (m, 7H), 3.58 (s, 3H), 3.01-3.13 (m, 2H), 2.66-2.78 (m, 3H), 2.51-2.57 (m, 1H), 2.44 (s, 3H). LCMS m/z (relative intensity) 669.3 [M+1]⁺ (30), 335.2 [M/2]⁺ (100).

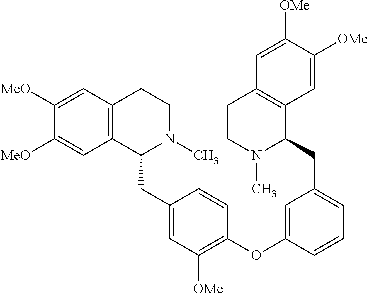 [SMILES: COc1cc2c(cc1OC)[C@@H](Cc1cccc(Oc3ccc(C[C@@H]4c5cc(OC)c(OC)cc5CCN4C)cc3OC)c1)N(C)CC2]

Bis-N-methyl-THIQ. White solid (87 mg, 67%): ¹H NMR (CDCl₃, 400 MHz) δ87.12-7.17 (m, 1H), 6.77-6.80 (m, 3H), 6.65-6.73 (m, 3H), 6.56 (s, 1H), 6.54 (s, 1H), 6.14 (s, 1H), 6.02 (s, 1H), 3.84 (s, 3H), 3.82 (s, 3H), 3.72 (s, 3H), 3.70-3.75 (m, 2H), 3.63 (s, 3H), 3.56 (s, 3H), 3.13-3.20 (m, 4H), 2.73-2.86 (m, 6H), 2.57-2.62 (m, 2H), 2.55 (s, 3H), 2.51 (s, 3H). ¹³C NMR (CDCl₃, 100 MHz) δ 157.90, 150.81, 147.36, 147.26, 146.47, 146.30, 143.19, 141.91, 136.66, 129.17, 129.03, 126.17, 125.76, 124.06, 122.24, 120.40, 118.65, 114.51, 114.28, 111.24, 111.15, 111.03, 110.90, 64.78, 64.63, 55.90, 55.78, 55.72, 55.65, 55,47, 47.05, 46.74, 42.73, 42.64, 41.19, 41.03, 25.55, 25.48 ppm; the remaining peak was not detected and believed to overlap with the peaks at 111.15 ppm.

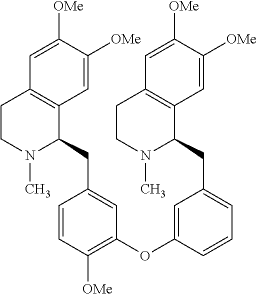 [SMILES: COc1cc2c(cc1OC)[C@@H](Cc1cccc(Oc3cc(C[C@@H]4c5cc(OC)c(OC)cc5CCN4C)ccc3OC)c1)N(C)CC2]

Bis-N-methyl-THIQ. White solid (61 mg, 66%): ¹³C NMR (CDCl₃, 100 MHz) δ157.95, 149.71, 147.24, 146.42, 146.34, 144.35, 142.01, 133.04, 130.72, 129.30, 129.17, 129.02, 126.17, 125.99, 125.78, 123.94, 122.43, 118.40, 114.24, 113.56, 112.40, 111.16, 111.04, 110.87, 64.94, 64.73, 64.64, 56.02, 55.73, 55.61, 55.48, 55.26, 46.98, 46.73, 42.72, 42.66, 41.17, 40.40, 25.50, 25.43 ppm.

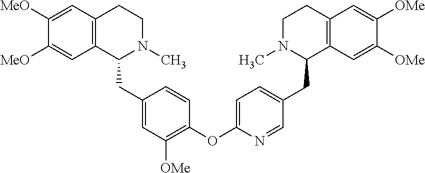 [SMILES: COc1cc2c(cc1OC)[C@@H](Cc1ccc(Oc3ccc(C[C@@H]4c5cc(OC)c(OC)cc5CCN4C)cc3OC)nc1)N(C)CC2]

Bis-N-methyl-THIQ. White solid (0.62 g, 58%): ¹H NMR (CDCl₃, 400 MHz) δ7.86 (d, J=2.0 Hz, 1H), 7.32 (dd, J=8.4, 2.4 Hz, 1H), 6.96 (d, J=8.0 Hz, 1H), 6.72-6.75 (m, 3H), 6.60-6.66 (m, 3H), 6.52 (s, 1H), 6.23 (s, 1H), 3.82 (s, 3H), 3.76-3.81 (m, 2H), 3.76 (s, 3H), 3.70 (s, 3H), 3.64 (s, 3H), 3.09-3.15 (m, 3H), 2.95-3.10 (m 1H), 2.82-2.90 (m, 3H), 2.65-2.75 (m, 4H), 2.51 (s, 3H), 2.49 (s, 3H). ¹³C NMR (CDCl₃, 100 MHz)) δ162.41, 157.71, 151.11, 148.15, 147.36, 146.71, 140.87, 140.64, 137.71, 135.77, 129.85, 129.07, 129.04, 128.71, 126.68, 122.39, 122.18, 114.40, 113.11, 111.73, 111.28, 110.65, 109.51, 64.49, 64.38, 55.84, 55.77, 55.17, 47.36, 47.16, 42.80, 42.78, 41.32, 37.37, 26.42, 25.68 ppm. LCMS m/z (relative intensity) 610.3 [M+1]⁺ (20), 305.7 [M/2]⁺ (100).

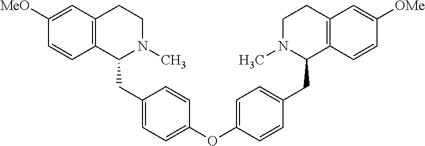 [SMILES: COc1ccc2c(c1)CCN(C)[C@@H]2Cc1ccc(Oc2ccc(C[C@@H]3c4ccc(OC)cc4CCN3C)cc2)cc1]

(R,R)-bis-N-methyl-THIQ. White solid (27 mg, 59%): NMR (CDCl₃, 400 MHz) δ7.02-7.26 (m, 5H), 6.86-6.93 (m, 3H), 6.60-6.67 (m, 6H), 3.73-3.81 (m, 8H), 3.08-3.19 (m, 4H), 2.61-2.86 (m, 8H), 2.50 (s, 6H). ¹³C NMR (CDCl₃, 100 MHz) δ157.63, 155.52, 151.11, 148.15, 147.36, 146.71, 140.87, 140.64, 137.71, 135.77, 129.85, 129.07, 129.04, 128.71, 126.68, 122.39, 122.18, 114.40, 113.11, 111.73, 111.28, 110.65, 109.51, 64.49, 64.38, 55.84, 55.77, 55.17, 47.36, 47.16, 42.80, 42.78, 41.32, 37.37, 26.42, 25.68 ppm. LCMS m/z (relative intensity) 549.2 [M+1]⁺ (100).

### In Vitro Screening of Anti-Filovirus Activity

Methods: Ebola virus infection assay.

All analogs herein were tested using an in vitro assay for inhibition of Ebola virus infection activity. All assays were performed with wild type (WT) Ebola virus, Mayinga strain. The infected cells were visualized by immunostaining with a specific antibody.

All assays used Huh7 cells, a cell line that was derived from a human liver carcinoma. These cells were chosen because the liver is a site for Ebola virus replication and a primary driver of pathogenesis. Therefore, a treatment for Ebola virus disease should show efficacy in the liver.

The procedure for the wild type Ebola virus infection assay is as follows:
1. In the BSL-2 tissue culture room, Huh7 cells were plated on 384 well plates with complete medium. 6,000 cells/well were dispensed. The volume of medium per well was kept at 25 μL. Prior to drug pre-incubation, cells were grown for 24 hours in a humidified incubator at 37° C. with 5% CO₂.
2. At the BSL-2 laboratory, all test compounds as well as tetrandrine were dissolved to 10 mM in DMSO and further diluted to 40 μM in complete medium. DMSO only was diluted to 0.4% in complete medium.
3. To the cell culture plates prepared in step 1, 25 μL, of diluted test compounds or DMSO were added to the last column (column 12 or 23) of each dilution series. Two-times dilution series were made from the last column (column 12 or 23) to the first column (column 2 or 13) by pipetting. Each compound was tested in triplicates.
4. Materials including the cell culture plates were transferred to the BSL-4 laboratory at the same time as the personnel were entering the BSL-4 laboratory to perform the following assay.
5. An appropriate amount of stock virus (wild type Ebola virus, Mayinga strain) was retrieved from the freezer and thawed at room temperature.
6. The virus was diluted 5,000 times by complete medium and 25 μL of diluted virus was added to each well of cell culture plates, which was pre-incubated with compounds. These plates were incubated for 24 hours in a humidified incubator at 37° C. with 5% CO₂.
7. After the incubation period, the plates were removed from the incubator and inactivated by immersing in 10% neutral buffered formalin. They were packaged in heat sealed bags, filled with enough 10% formalin to cover the plates. It was ensured that all the wells of the plates were completely filled with formalin upon immersion. The sealed bags were stored in the 4° C.±5° C. refrigerator overnight in the BSL-4 laboratory.
8. The sealed bags with plates were passed out of the BSL-4 laboratory via the chemical dunk tank.
9. The formalin inside the bags was disposed in a properly labeled hazardous waste container, and the plates were washed by dipping 3 times in 1× PBS.
10. 25 μL of 0.1% Triton X-100 in PBS was added to each well in the plates for permeabilization. After 10 minute incubation at room temperature, the plates were washed 3 times by 1× PBS.
11. 25 μL of 3.5% BSA in PBS was added to each well in the plates for blocking. After 1 hour incubation at room temperature, the plates were washed 3 times by 1× PBS.
12. Anti-Ebola virus glycoprotein antibody was diluted 1,000 times by 3.5% BSA for primary immune staining. 25 uL of the diluted antibody was added to each well in the plates. After 3 hour incubation at room temperature and additional overnight incubation in the 4° C. refrigerator, the plates were washed 3 times by 1× PBS.
13. Alexa Fluor 488 goat anti-mouse IgG antibody was diluted 1,000 times by 3.5% BSA for secondary immune staining. 25 μL of the diluted antibody was added to each well in the plates. After 1 hour incubation at room temperature, the plates were washed 3 times by 1× PBS.
14. 30 μL of Hoechst33342 dye (5,000 times diluent in 1× PBS) was added to each well. The plates were incubated overnight in the 4° C. refrigerator.
15. The plates were imaged by a Nikon Ti Eclipse inverted microscope using blue and green fluorescence channels to detect cell nuclei and infected cells, respectively.
16. Images were processed by CellProfiler cell image analysis software using an HTS analysis pipeline developed in the laboratory.

Data analysis was conducted according to the following procedure:
1. Raw data for cell nuclei numbers and infected cell numbers were transferred to a Microsoft Excel Spreadsheet.
2. Infection efficiencies were calculated by dividing the number of infected cells by total numbers of nuclei and normalizing to an average efficiency of mock-treated cells in the same plates.
3. Normalized infection rates were plotted with standard deviation versus compound doses in GraphPad Prism software.
4. Dose-response curves were drawn using a non-linear regression analysis.
5. IC50 value was calculated by the software for each compound.

All analogs herein were tested, in addition to tetrandrine. Each data point is from a set of 3 replicates with standard deviations indicated by error bars. The experiment was performed at one time. Curve fitting used log inhibitor vs response fit or for when lower asymptotes were not attained, a competitive enzyme inhibition curve was used in GraphPad Prism. An IC₅₀ value (concentration of compound giving 50% inhibition of virus infection) was calculated from each curve, and these values are shown in Tables 1, 2 and 3.

### Analysis of Anti-L-Type Channel Activity

All analogs were tested using an in vitro assay for inhibition of L-type calcium channel activity. The assays were performed using HEK293 cells stably overexpressing all subunits of L-type channel CaV1.2, which were gifted by Dr. Richard B. Silverman. See, Soosung Kang, Garry Cooper, Sara F. Dunne, Brendon Dusel, Chi-Hao Luan, D. James Surmeier and Richard B. Silverman. 2012, CaV1.3-selective L-type Calcium Channel Antagonists as Potential New Therapeutics for Parkinson's Disease, Nat. Commun. 7:11720. The cells were stained by a calcium indicator dye, Calcium 6, and stimulated by potassium chloride (KCl), which was reported to activate L-type channel but not two-pore channels. See, Edmund Naylor, Abdelilah Arredouani, Sridhar R Vasudevan, Alexander M Lewis, Raman Parkesh, Akiko Mizote, Daniel Rosen, Justyn M Thomas, Minoru Izumi, A Ganesan, Antony Galione and Grant C Churchill. 2009, Identification of a Chemical Probe for NAADP by Virtual Screening, Nat. Chem. Biol. 5:220-226.

The procedure for the L-type channel assay is as follows:
1. 384 well black plates were coated with Geltrex diluted in DMEM by incubating at 37° C. for 1 hour. After additional 1 hour incubation at room temperature, Geltrex was removed. This coating is to enhance cell attachment.
2. In the BSL-2 laboratory, HEK293 cells overexpressing CaV1.2 were plated on the coated plates with complete medium. 5,000 cells/well were plated. The volume of medium per well was kept at 25 μL. Cells were grown overnight in a humidified incubator at 37° C. with 5% CO₂.
3. Culture medium was removed and 50 μL/well of fresh complete medium was added. Cells were incubated further overnight in a humidified incubator at 37° C. with 5% CO₂.
4. 25 μL/well of culture medium was removed and 25 μL/well of Calcium 6 (Molecular Devices, LLC) was added to each well. The plate was incubated for 1.5 hour in a humidified incubator at 37° C. with 5% CO₂.
5. All analogs as well as tetrandrine were diluted to 8 mM in Dimethyl sulfoxide (DMSO). Nimodipine and verapamil were diluted to 4 μM and 0.67 mM, respectively, as positive controls. Three-fold dilution series of all compounds were made using DMSO with 6 dose points.
6. 0.5 μL of the diluted compounds was added to each well in cell culture plates containing Calcium 6 and was mixed. The plate was incubated for 30 minutes at room temperature. For background measurement, 0.5 μL of 1 mM of nimodipine was also added in one well of each row. In the first well of each row, 0.5 μL of DMSO only was added.
7. Using PHERAstar FS plate reader equipped with automatic injectors, fluorescence of each well was measured 20 seconds after injecting 10 μL/well of 500 mM KCl (the final concentration of KCl was 83 mM).

Data analysis was conducted according to the following procedure:
1. Raw data for fluorescence intensities were transferred to a Microsoft Excel Spreadsheet.
2. Background fluorescence intensity, which was measured by nifedipine treatment, was subtracted from each fluorescence intensity.
3. The fluorescence intensities were normalized to those of untreated wells.
4. Normalized fluorescence intensities were plotted in GraphPad Prism software.
5. Dose-response curves for nimodipine and verapamil were drawn using a non-linear regression analysis to calibrate the assay.

All analogs have been tested, in addition to tetrandrine. Each data point is from a set of 2 replicates with standard deviations indicated by error bars. See Tables 1-3.

$$activity = \frac{signal treated cells}{signal untreated cells}$$

Accordingly, the present invention is directed at a series of acyclic bis-benzyl-tetrahydroisoquinoline analogs that are derivatives of the cyclic products tetrandrine (TETN) and cepharanthine (CEPH). The analogs indicate activity against filovirus infections.

What is claimed is:

1. A bis-benzyl-tetrahydroisoquinoline comprising the following Formula 1: 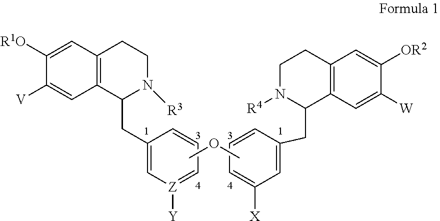
wherein R¹, R²=Me;
R¹ and V can combine to form a dioxolane ring,
R² and W can combine to form a dioxolane ring;
V, W═H, OMe;
R³, R⁴═H, Me, isopropyl, cyclopropyl, benzyl and C(O)—(CH₂)n-N-Me₂, n=1-3;
OR¹ can be replaced by a H;
X═H, OMe;
Z—Y═C—H, C—OMe, N; and
bi-aryl substitution: 1,4-1,4-; 1,4-1,3-; 1,3-1,3 isomers;
or a pharmaceutical salt thereof.

2. A bis-benzyl-tetrahydroisoquinoline comprising the following Formula 2: 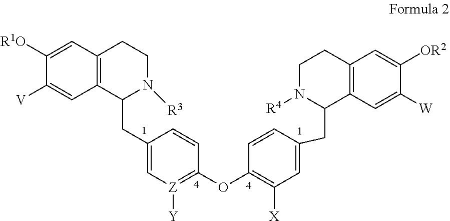
wherein R¹, R²=Me;
R¹ and V can combine to form a dioxolane ring;
R² and W combine to form a dioxolane ring;
V, W═H, OMe;
OR¹ can be replaced by a H;
R³, R⁴═H, Me, isopropyl, cyclopropyl, benzyl and —C(O)—(CH₂)n-NMe₂, n=1-3;
X═H, OMe; and
Z—Y═C—H, C—OMe, N;
or a pharmaceutical salt thereof.

3. The bis-benzyl-tetrahydroisoquinoline of claim 1 wherein:
R¹, R², R³ and R⁴=Me;
V, W, X and Y═H; and
Z═C

4. A bis-benzyl-tetrahydroisoquinoline comprising the following Formula 3: 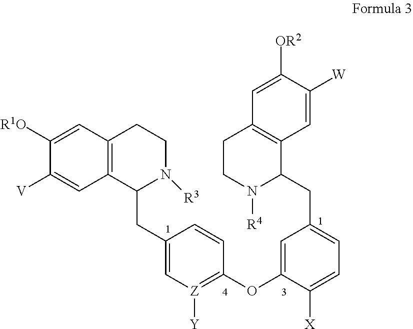
wherein R¹, R²=Me;
R¹ and V can combine to form a dioxolane ring;
R² and W can combine to form a dioxolane ring;
V, W═H, OMe;
R³, R⁴═H, Me, isopropyl, cyclopropyl, benzyl and C(O)—(CH₂)n-NMe₂, n=1-3;
X═H, OMe;
Z—Y═C—H, C—OMe, N;
or a pharmaceutical salt thereof.

5. A bis-benzyl-tetrahydroisoquinoline comprising the following Formula 4: 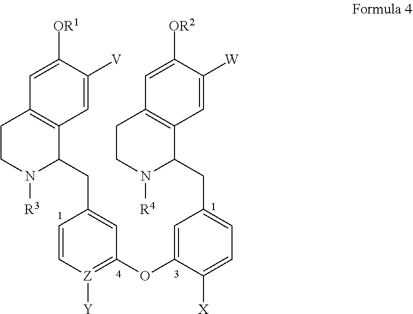
wherein R¹, R² =Me;
R¹ and V can combine to form a dioxolane ring;
R² and W can combine to form a dioxolane ring;
V, W═H, OMe;
R³, R⁴═H, Me, isopropyl, benzyl and C(O)—(CH₂)n-NMe₂, n=1-3;
X═H, OMe;
Z—Y═C—H, C—OMe, N;
or a pharmaceutical salt thereof.

6. A method for treating an individual infected with or exposed to a filovirus comprising administering to said individual as an active ingredient a compound of Formula 1: 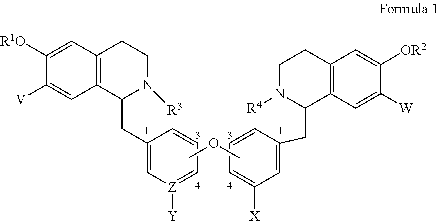
wherein R¹, R²=Me;
R¹ and V can combine to form a dioxolane ring,
R² and W can combine to form a dioxolane ring;
V, W═H, OMe;
R³, R⁴═H, Me, isopropyl, cyclopropyl, benzyl and C(O)—(CH₂)n-N-Me₂, n=1-3;
OR¹ can be replaced by a H;
X═H, OMe;
Z—Y═C—H, C—OMe, N; and
bi-aryl substitution: 1,4-1,4-; 1,4-1,3-; 1,3-1,3 isomers;
or a pharmaceutical salt thereof.

7. A method for treating an individual infected with or exposed to a filovirus comprising administering to said individual as an active ingredient a compound of Formula 2: 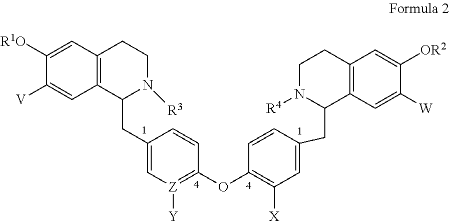
wherein R¹, R²=Me;
R¹ and V can combine to form a dioxolane ring;
R² and W combine to form a dioxolane ring;
V, W═H, OMe;
OR¹ can be replaced by a H;
R³, R⁴═H, Me, isopropyl, cyclopropyl, benzyl and —C(O)—(CH₂)n-NMe₂, n=1-3;
X═H, OMe; and
Z—Y=C—H, C—OMe, N;
or a pharmaceutical salt thereof.

8. The method of claim 7 wherein:
R¹, R², R³ and R⁴=Me;
V, W, X and Y═H; and
Z═C

9. A method for treating an individual infected with or exposed to a filovirus comprising administering to said individual as an active ingredient a compound of Formula 3: 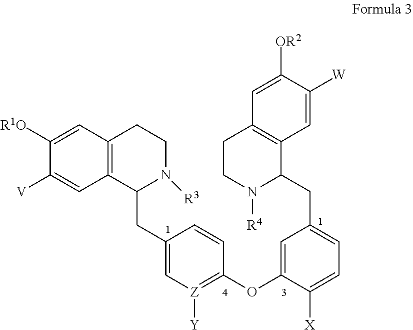
wherein R¹, R²=Me;
R¹ and V can combine to form a dioxolane ring;
R² and W can combine to form a dioxolane ring;
V, W═H, OMe;
R³, R⁴═H, Me, isopropyl, cyclopropyl, benzyl and C(O)—(CH₂)n-NMe₂, n=1-3;
X═H, OMe;
Z—Y═C—H, C—OMe, N;
or a pharmaceutical salt thereof.

10. A method for treating an individual infected with or exposed to a filovirus comprising administering to said individual as an active ingredient a compound of Formula 4: 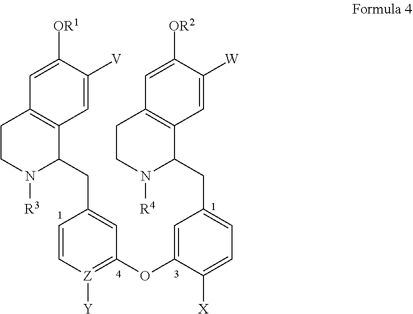
wherein R¹, R²=Me;
R¹ and V can combine to form a dioxolane ring;
R² and W can combine to form a dioxolane ring;
V, W═H, OMe;
R³, R⁴═H, Me, isopropyl, benzyl and C(O)—(CH₂)n-NMe₂, n=1-3;
X═H, OMe;
Z—Y═C—H, C—OMe, N;
or a pharmaceutical salt thereof.
